(19) United States
(12) Patent Application Publication
(10) Pub. No.: US 2011/0269688 A1
(43) Pub. Date: Nov. 3, 2011

(54) Genetic Alterations Associated with Schizophrenia and Methods of Use Thereof for the Diagnosis and Treatment of the Same

(76) Inventors: Hakon Hakonarson, Malvern, PA (US); Joseph Glessner, Turnersville, NJ (US); Struan F.A. Grant, Philadelphia, PA (US)

(21) Appl. No.: 13/108,652

(22) Filed: May 16, 2011

Related U.S. Application Data
(63) Continuation-in-part of application No. PCT/US09/64652, filed on Nov. 16, 2009.
(60) Provisional application No. 61/114,956, filed on Nov. 14, 2008.

(51) Int. Cl. A61K 31/454; G01N 33/53; C12Q 1/02; C12N 15/63; A61P 25/18; C12N 1/00; C07H 21/00; A61K 31/4015; A61K 38/02; C12Q 1/68; C12N 5/10

(52) U.S. Cl. 514/17.5; 435/6.11; 436/501; 435/29; 435/320.1; 435/325; 435/419; 435/243; 536/23.1; 514/424; 514/326

(57) ABSTRACT

Compositions and methods for the detection and treatment of schizophrenia are provided.

This application claims priority to PCT/US09/64652 filed Nov. 16, 2009 which in turn claims priority to U.S. Provisional Application 61/114,956 filed Nov. 14, 2008, the entire contents of each being incorporated herein by reference as though set forth in full.

### FIELD OF THE INVENTION

This invention relates to the fields of genetics and the diagnosis and treatment of schizophrenia. More specifically, the invention provides nucleic acids comprising copy number variations (CNVs) which are associated with the schizophrenia phenotype and methods of use thereof in diagnostic and therapeutic applications.

### BACKGROUND OF THE INVENTION

Several publications and patent documents are cited throughout the specification in order to describe the state of the art to which this invention pertains. Each of these citations is incorporated herein by reference as though set forth in full.

Schizophrenia is a chronic, severe, and disabling brain disorder that affects about 1.1 percent of the U.S. population. People with schizophrenia sometimes hear voices others don't hear, believe that others are broadcasting their thoughts to the world, or become convinced that others are plotting to harm them. These experiences can make them fearful and withdrawn and cause difficulties when they try to have relationships with others.

People with schizophrenia may not make sense when they talk, may sit for hours without moving or talking much, or may seem perfectly fine until they talk about what they are really thinking. Because many people with schizophrenia have difficulty holding a job or caring for themselves, the burden on their families and society is significant as well.

Available treatments can relieve many of the disorder's symptoms, but most people who have schizophrenia must cope with some residual symptoms as long as they live. Clearly, a need exists for improved compositions and methods for the diagnosis and treatment of this devastating neuronal disorder.

### SUMMARY OF THE INVENTION

In accordance with the present invention, a method for detecting a propensity for developing schizophrenia in a patient in need thereof is provided. An exemplary method entails detecting the presence of at least one CNV containing nucleic acid in a target polynucleotide wherein if said CNV is present, said patient has an increased risk for developing schizophrenia, wherein said CNV containing nucleic acid is selected from the group of CNVs that are either exclusive to, or significantly overrepresented in schizophrenia. See Tables 2 and 3). In another embodiment of the invention, a method for identifying agents which alter neuronal signaling and/or morphology is provided. Such a method comprises providing cells expressing at least one of the CNVs listed above (step a); providing cells which express the cognate wild type sequences corresponding to the CNV (step b); contacting the cells from each sample with a test agent and analyzing whether said agent alters neuronal signaling and/or morphology of cells of step a) relative to those of step b), thereby identifying agents which alter neuronal signaling and morphology. Methods of treating schizophrenic patients via administration of pharmaceutical compositions comprising agents identified using the methods described herein are also encompassed by the present invention.

The invention also provides at least one isolated schizophrenia related CNV-containing nucleic acid selected from the group that are either exclusive to, or significantly overrepresented in schizophrenia (see Table 2, Table 3, Table 4, Table 5 and Table 7). Such CNV containing nucleic acids may optionally be contained in a suitable expression vector for expression in neuronal cells. Alternatively, they may be immobilized on a solid support.

According to yet another aspect of the present invention, there is provided a method of treating schizophrenia in a patient determined to have at least one prescribed single nucleotide polymorphism indicative of the presence of a schizophrenia associated copy number variation, as described hereinbelow, by administering to the patient a therapeutically effective amount of at least one member of the piracetam family of nootropic agents. This method provides a test and treat paradigm, whereby a patient's genetic profile is used to personalize treatment with therapeutics targeted towards specific neurophysiological defects found in individuals exhibiting schizophrenia. Such a test and treat model may benefit up to 50% of patients with schizophrenia with greater efficacy and fewer side effects than non-personalized treatment.

### BRIEF DESCRIPTIONS OF THE DRAWINGS

FIG. 1: A web-browser view of significant CNVs, including GRIK5 (glutamate receptor, ionotropic, kainate 5, NTS (neurotensin), GRM5 (glutamate receptor, metabotropic 5) all of which are highly overrepresented in and associate with schizophrenia. To address the potential biological roles of the CNVs that were either associated with or overrepresented in schizophrenia, we performed Functional Annotation Clustering (FAC) of all the genes listed using the DAVID Bioinformatics Database. We observed that deleted genes classified with GO term ionotropic glutamate receptor activity (p=5.8×10⁻⁴) and the Neuroactive Ligand-Receptor Interaction by Kegg pathway (p=5.5×10⁻³) had significant enrichment among these schizophrenia candidate genes, which have striking biological relevance to schizophrenia. Genes in the ionotropic glutamate receptor activity GO category include GRIK1 (glutamate receptor, ionotropic, kainate 1), GRIA4 (glutamate receptor, ionotrophic, ampa 4), GRIN3A (glutamate receptor, ionotropic, n-methyl-d-aspartate 3a), and GRIK5 (glutamate receptor, ionotropic). The twelve associated genes in the Neuroactive Ligand-Receptor Interaction pathway include TACR3 (tachykinin receptor 3), GRIK1 (glutamate receptor, ionotropic, kainate 1), FSHR (follicle stimulating hormone receptor), GRIA4 (glutamate receptor, ionotrophic, ampa 4), GRIN3A (glutamate receptor, ionotropic, n-methyl-d-aspartate 3a), GABRG2 (gamma-aminobutyric acid (gaba) a receptor, gamma 2), LEPR (leptin receptor, TRH thyrotropin-releasing hormone), GRIK5 (glutamate receptor, ionotropic, kainate 5, NTS neurotensin), GRM5 (glutamate receptor, metabotropic 5), and MC4R (melanocortin 4 receptor).

FIG. 2. Eigenstrat Analysis of Genotype Bias A) Eigenstrat Principal Components Analysis of genotypes provided on dbGap showing three clear modes of clustering bias due to processing samples in batches. B) Eigenstrat Principal Components Analysis of genotypes generated at CHOP based on a single run of APT with a majority of samples falling in the region x>−0.01 and y<0.02 and few outliers due to ethnicity admixture.

FIG. 3. Affymetrix Genotyping console Canary CNV Call Viewed Heat Map for a Subset of Schizophrenia Case Deletions of 22q11.2

FIG. 4. Affymetrix Genotyping Console Browser Showing Log2Ratio of Schizophrenia Cases Deleted 3′ of CACNA1B on 9q34.3 and on RET on 10q11.21.

FIG. 5. The Number of CNV Calls Detected for Each Sample in Case and Control Sets. The distribution of CNV calls per individual in the discovery case:control CNV association.

FIG. 6. Examples of CNV observance based on B-allele frequency (BAF) and Log R Ratio (LRR).

FIG. 7. Frequency of Copy Number Variations Observed in Study Subjects. Red: Schizophrenia Case Deletion, Blue: Schizophrenia Case Duplication, Black: Schizophrenia Control Deletion, Purple: Schizophrenia Control Duplication. Maximum value displayed is 0.2 to make low frequency CNV, which is the majority of loci, visible.

FIG. 8. 16q22.1 Deletions found overrepresented in 30 independent schizophrenia cases. Affymetrix SNP and CN probe coverage shown with blue lines in two separate tracks. Schizophrenia cases with deletions and their CNV call boundaries shown in red lines. The schizophrenia cases of our 1,557 cases population run on Affymetrix 6.0 are shown in comparison to our control cohort of 3,485 showing overrepresentation in cases. ISC case and control CNV profiles also show overrepresentation. Note that duplications are conversely underrepresented in the schizophrenia cases (5) versus controls (27).

### DETAILED DESCRIPTION OF THE INVENTION

Schizophrenia is a devastating mental disorder characterized by reality distortion. Common features are positive symptoms of hallucinations, delusions, disorganized speech and abnormal thought process, negative symptoms of social deficit, lack of motivation, anhedonia and impaired emotion processing, and cognitive deficits with occupational dysfunction. Onset of symptoms typically occurs in late adolescence or early adulthood, with approximately 1.5% of the population affected¹.

Previous studies have associated various CNVs with schizophrenia including deletions of 22q11.2⁴, NRXN1⁵, APBA2⁵, and CNTNAP2⁶. However, each of these CNV is rare and they account for a relatively small proportion of the overall genetic risk in schizophrenia.

Recent reports have emphasized large rare CNVs impacting many different genes enriched in neurodevelopmental pathways⁷⁻⁹. Specifically, novel deletions and duplications of genes were reportedly observed in 15% of cases versus 5% of controls (P=0.0008) 9. However, a study of CNVs in Chinese schizophrenia patients detected no significant difference in rare CNVs between cases and controls¹⁰. Another study of 1,013 cases and 1,084 controls of European ancestry also failed to find more rare CNVs >100 kb in cases or enrichment for neurodevelopmental pathways¹¹. Specific loci exhibiting runs of homozygosity (ROHs) in schizophrenia cases have been associated³⁷. Significant association of de novo CNVs with schizophrenia (P=7.8×10⁻⁴) was found and were more frequent in sporadic cases than in controls³⁸.

We performed a genome-wide search for copy number variation (CNV) association to the schizophrenia phenotype. The study cohort included multiplex schizophrenia families where all subjects have been phenotyped by Dr. Deborah Levy at McLean University in Boston or by colleagues under her supervision. See Example I. The DNA samples obtained from Dr. Levy were genotyped using the HumanHap550K CNV chip platform from Illumina. To determine the potential contribution of the CNVs observed to associate with schizophrenia, we identified a matched control group from Philadelphia (available at CHOP) for comparison. The data quality was strictly filtered based on a call rate exceeding 98%. The populations of cases and controls were closely stratified based on Ancestry Informative Markers (ATMs) clustering, a standard deviation of normalized intensity below 0.35, low waviness of intensity corresponding with GC content, and a maximum count of 40 CNVs per individual. This resulted in 136 schizophrenia cases, 225 unaffected parents/siblings and 1338 disease-free control subjects without schizophrenia who had no evidence of neurological disease. Utilizing a Hidden Markov Model (HMM) approach implemented by the software program PennCNV developed by Penn and CHOP investigators (Wang et al, 2007), the most probable CNV state is reported for a contiguous sequence of CNVs for each individual sample in the Tables provided below.

In additional studies, the study cohort included 1,206 schizophrenia cases and 1,378 neurologically normal controls that were genotyped on the Affymetrix 6.0 array from the Genetic Association Information Network (GAIN)¹². We downloaded the data files from dbGaP (ncbi.nlm.nih.gov/gap Study: phs000021.v2.p1) and analyzed them for CNV associations. This project, also known as Molecular Genetics of Schizophrenia (MGS) has previously reported linkage to 8p23.3-p21.2 and 11p13.1-q14.1¹³ and association of FGFR2 in a GWAS¹⁴⁻¹⁵, but failed to associate previously reported candidate genes¹⁶ and found novel association of common genotype variants on 6p22.1¹⁷. In addition, 351 schizophrenia cases and 2,107 control subjects from the University of Pennsylvania were genotyped on the Affymetrix 6.0 array at CHOP. Control subjects were recruited by health studies of high HDL cholesterol, coronary angiography, and heart transplant at the University of Pennsylvania. The average age was 62 years and no subjects displayed major psychoses. Samples from these sources were divided in a discovery cohort of 977 cases and 2,000 controls and a replication cohort of 580 schizophrenia cases and 1,485 controls. Bias of contribution to specific loci was monitored between these two sample sources. See Example 2.

All patients were diagnosed with schizophrenia based on DSM-IV (Diagnostic and Statistical Manual of Mental Disorders)¹⁸. This comprehensive evaluation of schizophrenia related criteria encompasses the variable presentations and characteristics of schizophrenia to form robust inclusion criteria.

The CNVs identified herein provide new targets for the development of efficacious therapeutic agents for the diagnosis and treatment of schizophrenia.

### Definitions:

A “copy number variation (CNV)” refers to the number of copies of a particular gene in the genotype of an individual. CNVs represent a major genetic component of human phenotypic diversity. Susceptibility to genetic disorders is known to be associated not only with copy number variations (CNV), but also with structural and other genetic variations, including CNVs. A CNV represents a copy number change involving a DNA fragment that is ˜1 kilobases (kb) or larger (Feuk et al. 2006 Nature. 444:444-54.). CNVs described herein do not include those variants that arise from the insertion/deletion of transposable elements (e.g., ˜6-kb KpnI repeats) to minimize the complexity of future CNV analyses. The term CNV therefore encompasses previously introduced terms such as large-scale copy number variants (LCVs; lafrate et al. 2004, Nature Genetics 36: 949-51), copy number polymorphisms (CNPs; Sebat et al. 2004 Science 305:525-8.), and intermediate-sized variants (ISVs; Tuzun et al. 2006 Genome Res. 16: 949-961), but not retroposon insertions.

A “single nucleotide polymorphism (SNP)” refers to a change in which a single base in the DNA differs from the usual base at that position. These single base changes are called SNPs or “snips.” Millions of SNP's have been cataloged in the human genome. Some SNPs such as that which causes sickle cell are responsible for disease. Other SNPs are normal variations in the genome.

The term “genetic alteration” which encompasses a CNV or SNP as defined above, refers to a change from the wild-type or reference sequence of one or more nucleic acid molecules. Genetic alterations include without limitation, base pair substitutions, additions and deletions of at least one nucleotide from a nucleic acid molecule of known sequence.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

The phrase “consisting essentially of when referring to a particular nucleotide or amino acid means a sequence having the properties of a given SEQ ID NO:. For example, when used in reference to an amino acid sequence, the phrase includes the sequence per se and molecular modifications that would not affect the functional and novel characteristics of the sequence.

“Target nucleic acid” as used herein refers to a previously defined region of a nucleic acid present in a complex nucleic acid mixture wherein the defined wild-type region contains at least one known nucleotide variation which may or may not be associated with schizophrenia. The nucleic acid molecule may be isolated from a natural source by cDNA cloning or subtractive hybridization or synthesized manually. The nucleic acid molecule may be synthesized manually by the triester synthetic method or by using an automated DNA synthesizer.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁻⁶-fold purification of the native message. Thus, purification of at least one order of magnitude, preferably two or three orders, and more preferably four or five orders of magnitude is expressly contemplated.

The term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to any schizophrenia specific marker gene or nucleic acid, but does not hybridize to other nucleotides. Also polynucleotide which “specifically hybridizes” may hybridize only to a neurospecific specific marker, such an schizophrenia-specific marker shown in the Tables contained herein. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning,

Cold Spring Harbor Laboratory (1989):

Tₘ=81.5″C+16.6 Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57″C. The Tₘ of a DNA duplex decreases by 1-1.5″C with every 1% decrease in homology. Thus, targets with greater than about 75% sequence identity would be observed using a hybridization temperature of 42″C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “oligonucleotide,” as used herein is defined as a nucleic acid molecule comprised of two or more ribo- or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20 nucleotides in length.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the schizophrenia specific marker nucleic acid molecule such that the latter is transcribed into mRNA. Host cell machinery then translates mRNA into a polypeptide.

Those skilled in the art will recognize that a nucleic acid vector can contain nucleic acid elements other than the promoter element and the schizophrenia specific marker gene nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

“Sample” or “patient sample” or “biological sample” generally refers to a sample which may be tested for a particular molecule, preferably a schizophrenia specific marker molecule, such as a marker shown in the tables provided below. Samples may include but are not limited to cells, body fluids, including blood, serum, plasma, urine, saliva, tears, pleural fluid and the like.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, snRNA, antisense oligonucleotides, peptides, peptide/DNA complexes, and any nucleic acid based molecule which exhibits the capacity to modulate the activity of the CNV containing nucleic acids described herein or their encoded proteins. Agents are evaluated for potential biological activity by inclusion in screening assays described hereinbelow.

### Methods of Using Schizophrenia-Associated CNVS for Diagnosing a Propensity for the Development of Schizophrenia

Schizophrenia-related-CNV containing nucleic acids, including but not limited to those listed in the Tables provided below may be used for a variety of purposes in accordance with the present invention. Schizophrenia-associated CNV containing DNA, RNA, or fragments thereof may be used as probes to detect the presence of and/or expression of schizophrenia specific markers. Methods in which schizophrenia specific marker nucleic acids may be utilized as probes for such assays include, but are not limited to: (1) in situ hybridization; (2) Southern hybridization (3) northern hybridization; and (4) assorted amplification reactions such as polymerase chain reactions (PCR).

Further, assays for detecting schizophrenia-associated CNVs may be conducted on any type of biological sample, including but not limited to body fluids (including blood, urine, serum, gastric lavage), any type of cell (such as brain cells, white blood cells, mononuclear cells) or body tissue.

From the foregoing discussion, it can be seen that schizophrenia-associated CNV containing nucleic acids, vectors expressing the same, schizophrenia CNV containing marker proteins and anti-schizophrenia specific marker antibodies of the invention can be used to detect schizophrenia associated CNVs in body tissue, cells, or fluid, and alter schizophrenia CNV containing marker protein expression for purposes of assessing the genetic and protein interactions involved in the development of schizophrenia.

In most embodiments for screening for schizophrenia-associated CNVs, the schizophrenia-associated CNV containing nucleic acid in the sample will initially be amplified, e.g. using PCR, to increase the amount of the templates as compared to other sequences present in the sample. This allows the target sequences to be detected with a high degree of sensitivity if they are present in the sample. This initial step may be avoided by using highly sensitive array techniques that are becoming increasingly important in the art.

Alternatively, new detection technologies can overcome this limitation and enable analysis of small samples containing as little as 1 μg of total RNA. Using Resonance Light Scattering (RLS) technology, as opposed to traditional fluorescence techniques, multiple reads can detect low quantities of mRNAs using biotin labeled hybridized targets and anti-biotin antibodies. Another alternative to PCR amplification involves planar wave guide technology (PWG) to increase signal-to-noise ratios and reduce background interference. Both techniques are commercially available from Qiagen Inc. (USA).

Thus any of the aforementioned techniques may be used to detect or quantify schizophrenia-associated CNV marker expression and accordingly, diagnose schizophrenia.

### Kits and Articles of Manufacture

Any of the aforementioned products can be incorporated into a kit which may contain a schizophrenia-associated CNV specific marker polynucleotide or one or more such markers immobilized on a Gene Chip, an oligonucleotide, a polypeptide, a peptide, an antibody, a label, marker, or reporter, a pharmaceutically acceptable carrier, a physiologically acceptable carrier, instructions for use, a container, a vessel for administration, an assay substrate and/or enzyme, or any combination thereof.

### Methods of Using Schizophrenia-Associated CNVs/SNPs for Development of Therapeutic Agents

Since the CNVs identified herein have been associated with the etiology of schizophrenia, methods for identifying agents that modulate the activity of the genes and their encoded products containing such CNVs should result in the generation of efficacious therapeutic agents for the treatment of this condition.

As can be seen from the data provided in the Tables below, several chromosomes contain regions which provide suitable targets for the rational design of therapeutic agents which modulate their activity. Specific organic molecules can thus be identified with capacity to bind to the active site of the proteins encoded by the CNV containing nucleic acids based on conformation or key amino acid residues required for function. A combinatorial chemistry approach will be used to identify molecules with greatest activity and then iterations of these molecules will be developed for further cycles of screening. In certain embodiments, candidate agents can be screening from large libraries of synthetic or natural compounds. Such compound libraries are commercially available from a number of companies, including but not limited to Maybridge Chemical Co., (Trevillet, Cornwall, UK), Comgenex (Princeton, N.J.), Microsour (New Milford, Conn.) Aldrich (Milwaukee, Wis.) Akos Consulting and Solutions GmbH (Basel, Switzerland), Ambinter (Paris, France), Asinex (Moscow, Russia) Aurora (Graz, Austria), BioFocus DPI (Switzerland), Bionet (Camelford, UK), Chembridge (San Diego, Calif.), Chem Div (San Diego, Calif.). The skilled person is aware of other sources and can readily purchase the same. Once therapeutically efficacious compounds are identified in the screening assays described herein, they can be formulated in to pharmaceutical compositions and utilized for the treatment of schizophrenia.

The polypeptides or fragments employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested.

Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells (such as described above) which have a nonfunctional or altered schizophrenia associated gene. These host cell lines or cells are defective at the polypeptide level. The host cell lines or cells are grown in the presence of drug compound. The rate of cellular metabolism of the host cells is measured to determine if the compound is capable of regulating the cellular metabolism in the defective cells. Host cells contemplated for use in the present invention include but are not limited to bacterial cells, fungal cells, insect cells, mammalian cells, and plant cells. The schizophrenia-associated CNV encoding DNA molecules may be introduced singly into such host cells or in combination to assess the phenotype of cells conferred by such expression. Methods for introducing DNA molecules are also well known to those of ordinary skill in the art. Such methods are set forth in Ausubel et al. eds., Current Protocols in Molecular Biology, John Wiley & Sons, NY, N.Y. 1995, the disclosure of which is incorporated by reference herein.

A wide variety of expression vectors are available that can be modified to express the novel DNA sequences of this invention. The specific vectors exemplified herein are merely illustrative, and are not intended to limit the scope of the invention. Expression methods are described by Sambrook et al. Molecular Cloning: A Laboratory Manual or Current Protocols in Molecular Biology 16.3-17.44 (1989). Expression methods in Saccharomyces are also described in Current Protocols in Molecular Biology (1989).

Suitable vectors for use in practicing the invention include prokaryotic vectors such as the pNH vectors (Stratagene Inc., 11099 N. Torrey Pines Rd., La Jolla, Calif. 92037), pET vectors (Novogen Inc., 565 Science Dr., Madison, Wis. 53711) and the pGEX vectors (Pharmacia LKB Biotechnology Inc., Piscataway, N.J. 08854). Examples of eukaryotic vectors useful in practicing the present invention include the vectors pRc/CMV, pRc/RSV, and pREP (Invitrogen, 11588 Sorrento Valley Rd., San Diego, Calif. 92121); pcDNA3.1/V5&His (Invitrogen); baculovirus vectors such as pVL1392, pVL1393, or pAC360 (Invitrogen); and yeast vectors such as YRP17, YIPS, and YEP24 (New England Biolabs, Beverly, Mass.), as well as pRS403 and pRS413 Stratagene Inc.); Picchia vectors such as pHIL-D1 (Phillips Petroleum Co., Bartlesville, Okla. 74004); retroviral vectors such as PLNCX and pLPCX (Clontech); and adenoviral and adeno-associated viral vectors.

Promoters for use in expression vectors of this invention include promoters that are operable in prokaryotic or eukaryotic cells. Promoters that are operable in prokaryotic cells include lactose (lac) control elements, bacteriophage lambda (pL) control elements, arabinose control elements, tryptophan (trp) control elements, bacteriophage T7 control elements, and hybrids thereof. Promoters that are operable in eukaryotic cells include Epstein Barr virus promoters, adenovirus promoters, SV40 promoters, Rous Sarcoma Virus promoters, cytomegalovirus (CMV) promoters, baculovirus promoters such as AcMNPV polyhedrin promoter, Picchia promoters such as the alcohol oxidase promoter, and Saccharomyces promoters such as the gal4 inducible promoter and the PGK constitutive promoter, as well as neuronal-specific platelet-derived growth factor promoter (PDGF), the Thy-1 promoter, the hamster and mouse Prion promoter (MoPrP), and the Glial fibrillar acidic protein (GFAP) for the expression of transgenes in glial cells.

In addition, a vector of this invention may contain any one of a number of various markers facilitating the selection of a transformed host cell. Such markers include genes associated with temperature sensitivity, drug resistance, or enzymes associated with phenotypic characteristics of the host organisms.

Host cells expressing the schizophrenia-associated CNVs of the present invention or functional fragments thereof provide a system in which to screen potential compounds or agents for the ability to modulate the development of schizophrenia. Thus, in one embodiment, the nucleic acid molecules of the invention may be used to create recombinant cell lines for use in assays to identify agents which modulate aspects of cellular metabolism associated with neuronal signaling and neuronal cell communication and structure. Also provided herein are methods to screen for compounds capable of modulating the function of proteins encoded by CNV containing nucleic acids.

Another approach entails the use of phage display libraries engineered to express fragment of the polypeptides encoded by the CNV containing nucleic acids on the phage surface. Such libraries are then contacted with a combinatorial chemical library under conditions wherein binding affinity between the expressed peptide and the components of the chemical library may be detected. U.S. Pat. Nos. 6,057,098 and 5,965,456 provide methods and apparatus for performing such assays.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacore.

Thus, one may design drugs which have, e.g., improved polypeptide activity or stability or which act as inhibitors, agonists, antagonists, etc. of polypeptide activity. By virtue of the availability of CNV containing nucleic acid sequences described herein, sufficient amounts of the encoded polypeptide may be made available to perform such analytical studies as x-ray crystallography. In addition, the knowledge of the protein sequence provided herein will guide those employing computer modeling techniques in place of, or in addition to x-ray crystallography.

In another embodiment, the availability of schizophrenia-associated CNV containing nucleic acids enables the production of strains of laboratory mice carrying the schizophrenia-associated CNVs of the invention. Transgenic mice expressing the schizophrenia-associated CNV of the invention provide a model system in which to examine the role of the protein encoded by the CNV containing nucleic acid in the development and progression towards schizophrenia. Methods of introducing transgenes in laboratory mice are known to those of skill in the art. Three common methods include: 1. integration of retroviral vectors encoding the foreign gene of interest into an early embryo; 2. injection of DNA into the pronucleus of a newly fertilized egg; and 3. the incorporation of genetically manipulated embryonic stem cells into an early embryo. Production of the transgenic mice described above will facilitate the molecular elucidation of the role that a target protein plays in various cellular metabolic, neuronal and cognitive processes. Such mice provide an in vivo screening tool to study putative therapeutic drugs in a whole animal model and are encompassed by the present invention.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

The alteration of genetic information may be foreign to the species of animal to which the recipient belongs, or foreign only to the particular individual recipient, or may be genetic information already possessed by the recipient. In the last case, the altered or introduced gene may be expressed differently than the native gene. Such altered or foreign genetic information would encompass the introduction of schizophrenia-associated CNV containing nucleotide sequences.

The DNA used for altering a target gene may be obtained by a wide variety of techniques that include, but are not limited to, isolation from genomic sources, preparation of cDNAs from isolated mRNA templates, direct synthesis, or a combination thereof.

A preferred type of target cell for transgene introduction is the embryonal stem cell (ES). ES cells may be obtained from pre-implantation embryos cultured in vitro (Evans et al., (1981) Nature 292:154-156; Bradley et al., (1984) Nature 309:255-258; Gossler et al., (1986) Proc. Natl. Acad. Sci. 83:9065-9069). Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of individual genes and their expression products is to use isolated schizophrenia-associated CNV genes as insertional cassettes to selectively inactivate a wild-type gene in totipotent ES cells (such as those described above) and then generate transgenic mice. The use of gene-targeted ES cells in the generation of gene-targeted transgenic mice was described, and is reviewed elsewhere (Froliman et al., (1989) Cell 56:145-147; Bradley et al., (1992) Bio/Technology 10:534-539).

Techniques are available to inactivate or alter any genetic region to a mutation desired by using targeted homologous recombination to insert specific changes into chromosomal alleles. However, in comparison with homologous extrachromosomal recombination, which occurs at a frequency approaching 100%, homologous plasmid-chromosome recombination was originally reported to only be detected at frequencies between 10⁻⁶ and 10⁻³. Nonhomologous plasmid-chromosome interactions are more frequent occurring at levels 10⁵-fold to 10² fold greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening of individual clones. Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodou-racil, (FIAU). By this counter selection, the number of homologous recombinants in the surviving transformants can be increased. Utilizing schizophrenia-associated CNV containing nucleic acid as a targeted insertional cassette provides means to detect a successful insertion as visualized, for example, by acquisition of immunoreactivity to an antibody immunologically specific for the polypeptide encoded by schizophrenia-associated CNV nucleic acid and, therefore, facilitates screening/selection of ES cells with the desired genotype.

As used herein, a knock-in animal is one in which the endogenous murine gene, for example, has been replaced with human schizophrenia-associated CNV containing gene of the invention. Such knock-in animals provide an ideal model system for studying the development of schizophrenia.

As used herein, the expression of a schizophrenia-associated CNV containing nucleic acid, fragment thereof, or an schizophrenia-associated CNV fusion protein can be targeted in a “tissue specific manner” or “cell type specific manner” using a vector in which nucleic acid sequences encoding all or a portion of schizophrenia-associated CNV are operably linked to regulatory sequences (e.g., promoters and/or enhancers) that direct expression of the encoded protein in a particular tissue or cell type. Such regulatory elements may be used to advantage for both in vitro and in vivo applications. Promoters for directing tissue specific proteins are well known in the art and described herein.

The nucleic acid sequence encoding the schizophrenia-associated CNV of the invention may be operably linked to a variety of different promoter sequences for expression in transgenic animals. Such promoters include, but are not limited to a prion gene promoter such as hamster and mouse Prion promoter (MoPrP), described in U.S. Pat. No. 5,877,399 and in Borchelt et al., Genet. Anal. 13(6) (1996) pages 159-163; a rat neuronal specific enolase promoter, described in U.S. Pat. Nos. 5,612,486, and 5,387,742; a platelet-derived growth factor B gene promoter, described in U.S. Pat. No. 5,811,633; a brain specific dystrophin promoter, described in U.S. Pat. No. 5,849,999; a Thy-1 promoter; a PGK promoter; a CMV promoter; a neuronal-specific platelet-derived growth factor B gene promoter; and Glial fibrillar acidic protein (GFAP) promoter for the expression of transgenes in glial cells.

Methods of use for the transgenic mice of the invention are also provided herein. Transgenic mice into which a nucleic acid containing the schizophrenia-associated CNV or its encoded protein have been introduced are useful, for example, to develop screening methods to screen therapeutic agents to identify those capable of modulating the development of schizophrenia.

### Pharmaceuticals and peptide Therapies

The elucidation of the role played by the schizophrenia associated CNVs described herein in neuronal signaling and brain structure facilitates the development of pharmaceutical compositions useful for treatment and diagnosis of schizophrenia. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

The following materials and methods are provided to facilitate the practice of Example 1.

### Illumina Infinium Assay.

We performed high-throughput, genome-wide SNP genotyping, using the InfiniumII HumanHap550 BeadChip technology (Illumina), at the Center for Applied Genomics at CHOP. Quantitative polymerase chain reaction (QPCR) may also be used to detect these aberrations. We used 750 ng of genomic DNA to genotype each sample, according to the manufacturer's guidelines. Single-base extension (SBE) uses a single probe sequence 50 by long that is designed to hybridize immediately adjacent to the SNP query site. After targeted hybridization to the bead array, the arrayed SNP locus-specific primers (attached to beads) were extended with a single hapten-labelled dideoxynucleotide in the SBE reaction. The haptens were subsequently detected by a multi-layer immunohistochemical sandwich assay, as recently described. The Illumina BeadArray Reader scanned each BeadChip at two wavelengths and created an image file. As BeadChip images were collected, intensity values were determined for all instances of each bead type, and data files were created that summarized intensity values for each bead type. These files consisted of intensity data that were loaded directly into Illumina's genotype analysis software, BeadStudio. A bead pool manifest created from the laboratory information management system (LIMS) database containing all the BeadChip data was loaded into BeadStudio along with the intensity data for the samples. BeadStudio used a normalization algorithm to minimize BeadChip to BeadChip variability. Once the normalization was complete, the clustering algorithm was run to evaluate cluster positions for each locus and to assign individual genotypes. Each locus was given an overall score, which was based on the quality of the clustering, and each individual genotype call was given a GenCall score. GenCall scores provided a quality metric that ranges from 0 to 1 assigned to every genotype called. GenCall scores were then calculated using information from the clustering of the samples. The location of each genotype relative to its assigned cluster determined its GenCall score.

### Illumina Infinium Assay for CNV Discovery

The genotype data content together with the intensity data provided by the genotyping array provides excellent confidence for CNV calls. The array platform used in this study provides a highly robust and reproducible SNP clustering due to the random placement of SNP specific beads with approximately 18-fold redundancy for each SNP. Using a SNP array provides allele frequency data which can be analyzed and more closely quality controlled for redundancy and high performance when compared to public databases. This establishes a more robust definition for normal diploid states than can be provided by aCGH technologies which are more variable due to batch processing issues. The genotype clustering establishes the probe performance at each locus for the expected heterozygous genotype state. Based on the hybridization efficiency, this may tend more to the DNP tagged Red range or the Biotin tagged Green range for any given locus. The normalization preformed to calculate B allele frequency (BAF) from theta adjusts the SNP specific range to a 0.5 expected value. This creates more continuous data since the heterozygous state is properly modeled based on extensive genotyping. Another key technical strength of our study is that the same array was typed at the same genotyping facility at the same time with the same cluster file for cases and controls. The data analysis is also standardized as described in the methods and CNVs are called with the same version of PennCNV¹².

### CNV Quality Control

458 samples were submitted for Illumina array typing by Deborah L. Levy, Ph.D. at the Mailman Research Center in McLean Hospital, affiliated with Harvard Medical School. We performed extensive Quality Control (QC) measures on our HumanHap550 GWAS data, where we included only high quality samples based on the following parameters: Call rate >98%, SD of normalized intensity (LRR) <0.35, adjustment for wave artifacts resulting from hybridization bias of low full length DNA quantity and proper balance of B-Allele Frequency (BAF). We also excluded samples with unusually high numbers of CNV calls because this can often reflect problematic DNA or arrays. Monozygotic twins or samples otherwise with cryptic relatedness were removed. Following QC, 136 Caucasian individuals with schizophrenia including 36 trios were analyzed with 1,338 controls.

### Statistical Analysis of CNVs

To call CNVs, we used the PennCNV algorithm (Wang et al. 2007), which combines multiple sources of information, including Log R Ratio (LRR) and B Allele Frequency (BAF) at each SNP marker, and SNP spacing and population frequency of the B allele to generate CNV calls from whole-genome SNP genotyping platforms. CNV frequency between cases and controls was evaluated at each SNP using a Fisher's exact test. We report statistical local minimums to narrow the association in reference to a region of nominal significance including SNPs residing within 1MB of each other. This leads to many significant regions with only one gene, an improvement over previous studies that implicated regions containing many genes. Resulting significant CNVRs were excluded if they met any of the following criteria: i) residing on telomere or centromere proximal cytobands; ii) arising in a peninsula of common CNV resulting in variation in boundary truncation of CNV calling; iii) being characterized by extremes in GC content which produces hybrization bias; iv) if included in the Database for Genomic Variants, or; v) contributing to multiple CNVRs. DAVID was used for gene clustering.

### CNV Validation by Visual Examination of BeadStudio Signal Plot

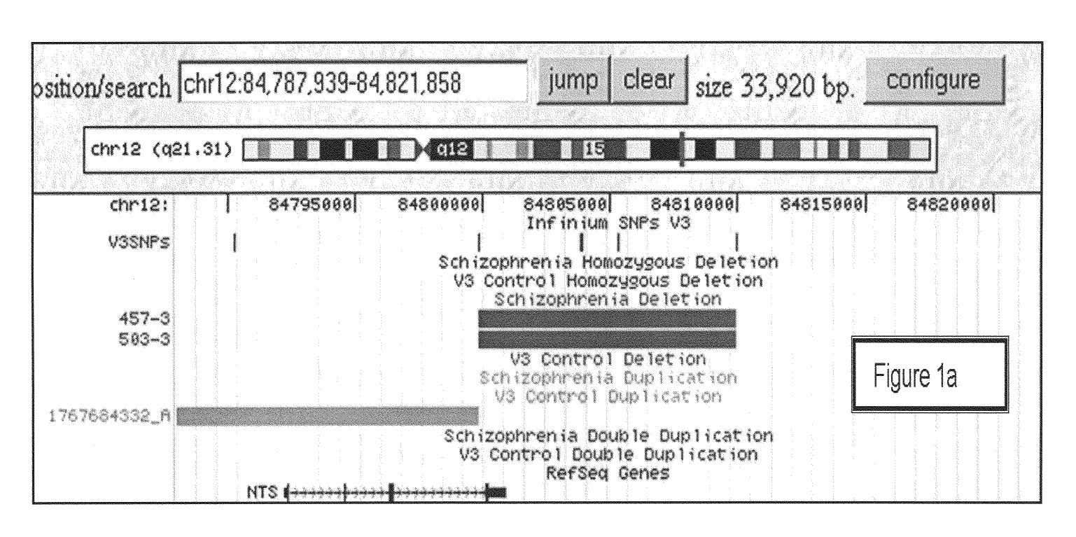

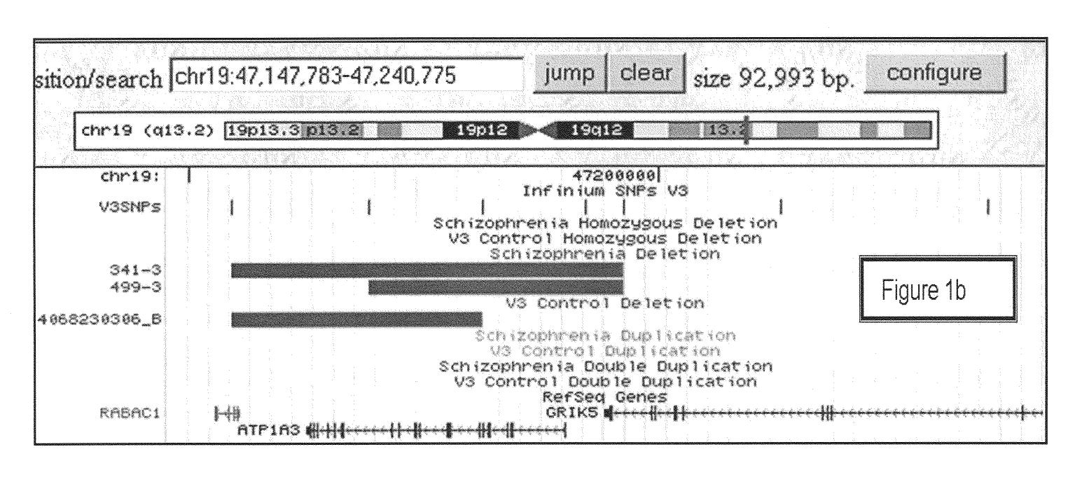

The Illumina BeadStudio software provides convenient visualization tools that allow the display of actual signal intensity data for the entire chromosome, with ability to zoom into a specified genomic region. Large CNV calls (typically those covered by >20 SNPs) can be easily visualized and confirmed in the BeadStudio software, based on the known signal characteristic for each copy number state (See FIG. 1 in¹²).

### Schizophrenia Diagnosis Inclusion Criteria

All subjects must give signed, informed consent.

Probands must have a consensus best-estimate DSM-IV (Diagnostic and Statistical Manual of Mental Disorders) diagnosis of SZ (schizophrenia) or of schizoaffective disorder with at least six months' duration of the “A” criteria for schizophrenia. Subjects must be over 18 years of age at interview, male or female. The informant should have known the subject for at least two years, be familiar with the psychiatric history, and have at least one hour of contact per week with the proband (close family members preferred).

Exclusion Criteria

Unable to give informed consent to all aspects of the study.

Unable to speak and be interviewed in English (to ensure validity of the interviews).

Psychosis is deemed secondary to substance use by the consensus diagnostic procedure because psychotic symptoms are limited to periods of likely intoxication or withdrawal, or there are persistent symptoms which are likely to be related to substance use (i.e., increasing paranoia after years of amphetamine use; symptoms limited to visual hallucinations after extensive hallucinogen use). The psychotic disorder is deemed secondary to a neurological disorder such as epilepsy based on the nature and timing of symptoms. For example, non-specific, non-focal EEG abnormalities are common in SZ, but subjects with psychosis that emerged in the context of temporal lobe epilepsy would be excluded.

Subjects with severe mental retardation (MR). Subjects with mild MR (IQ is greater than or equal to 55 or based on clinical and educational history) will be included, if SZ symptoms and history can be clearly established.

The examples set forth below are intended to illustrate certain embodiments of the invention. They are not intended to limit the invention in any way.

### EXAMPLE I

### Identification of CNVs which Associate with the Schizophrenic Phenotype

Schizophrenia is a late adolescent-onset psychiatric disease typically characterized by delusions, hallucinations and thought disturbances. We have confirmed association of DISC1, GRIA4, and CHN2 with schizophrenia. To determine if CNVs contribute to the development of schizophrenia, we performed extensive QC on Illumina550 data including call rate >98%, SD of normalized intensity (LRR) <0.35, low wave artifact correlating with GC content due to hybridization bias of low full length DNA quant −0.2<X<0.4, and proper balance of B-Allele Frequency (BAF). Following QC, 136 Caucasian individuals with schizophrenia including 36 trios were analyzed with 1,338 controls. Key Illumina array features for CNV include random placement of SNP specific beads on each array, 18 fold assay redundancy, and expected genotype color contrast to supplement intensity data. PennCNV (Wang et al, 2007) was used to call CNVs applying a Hidden Markov Model. CNV at each SNP was evaluated genome wide with chi square testing. Statistical local minimums were reported in reference to a region of nominal significance of SNPs residing within 1 MB. Associated regions were reviewed for call accuracy, lack of peninsulas created by boundary truncation, continuity of coverage, and compared with the Database for Genomic Variants. After review, 11 CNV regions (7 resided on genes) remained with at least 2 CNV cases. Genes with functional relevance to schizophrenia included NTS, GRIK5, and GRM5 (All p=8.5E-3). Functional clustering of independently associated results provided: ionotropic glutamate receptor activity (p=5.8E-4 GRIK1, GRIA4, GRIN3A, and GRIK5). We conclude that 6 genes harboring 9 CNVs (in 9 cases) in neurotransmission may account for a significant number of schizophrenia cases.

We first searched for replication of CNVs previously reported to associate with schizophrenia, including but not limited to DISC1, NPAS3, GRIA4, SEMA3A, CHN2 and NTF3. Table 1 displays previously reported genes that we could confirm through CNV association (DISC1 P=0.024; GRIA4, SEMA3A and CHN2 P=0.092). There was no evidence for association to the remaining genes that have previously been associated with schizophrenia using a candidate gene approach.

TABLE 1
Attempts to replicate CNVs previously linked with
schizophrenia (DISC1, GRIA4, CHN2, SEMA3A replicated)

| | | Gene | |
|---|---|---|---|
| Gene | Variation | Description | Region Impacted |
| DISC1 | 2 duplications | disrupted in | chr1: |
| | | schizophrenia 1 | 229831759-229905017 |
| | | isoform S | |
| GRIA4 | 1 deletion | glutamate | chr11: |
| | | receptor, | 104986621-105238570 |
| | | ionotrophic, | |
| | | AMPA 4 | |
| | | isoform | |
| CHN2 | 1 duplication | beta | chr7: 29486011-29520469 |
| | | chimerin | |
| | | isoform 1 | |
| SEMA3A | 1 duplication | semaphorin | chr7: 83425595-83662153 |
| | | 3A precursor | |

DICS1 was the only gene to replicate to a significant p value (P = 0.024).
The other genes showed a significant trend (P = 0.09) but were not significant, which may be due to sample size.

We next performed a CNV based whole genome CNV association to capture the most significant points in complex CNV overlap between case and control populations. A chi square statistic is applied to the CNV observance of deletion and duplication for each CNV. To present results in a non-redundant manner, statistical local minimums are reported in reference to regions of significance (p<0.05) where we incorporate all CNVs residing within 1 Mb of the most significant CNV. We identified regions of deletion (see Table 2) and duplication (see Table 3) CNVs in schizophrenia using this approach. The majority of genes identified are functionally linked with neuronal processes such as signaling and development that are highly relevant with respect to schizophrenia, including but not limited to the genes, NTS, GRIK5, and GRM5.

TABLE 2
Deletion CNVs in Schizophrenia: SNP based whole genome CNV
association analysis. Based on 136 Schizophrenia affected cases and 1338
controls. CNVs that are underlined are not found in unaffected subjects

| | | | Cases | Control |
|---|---|---|---|---|
| CNVR | Gene | P value | Loss | Loss |
| chr1: 194097653-194148082 | KCNT2, | 0.00182062 | 5 | 6 |
| | SLICK | | | |
| chr12: 84799874-84809923 | NTS | 0.008467635 | 2 | 0 |
| chr19: 47192213-47196345 | GRIK5 | 0.008467635 | 2 | 0 |
| chr3: 60564450-60565103 | FHIT | 0.008467635 | 2 | 0 |
| chr5: 78285889-78300797 | ARSB | 0.008467635 | 2 | 0 |
| chr13: 81402686-81416252 | SPRY2 | 0.008467635 | 2 | 0 |
| chr11: 88016449-88023261 | GRM5 | 0.008502244 | 2 | 0 |

TABLE 3
Duplication CNVs in Schizophrenia: SNP based whole
genome CNV association analysis

| | | | | Control |
|---|---|---|---|---|
| CNVR | Gene | P value | Cases Dup | Dup |
| chr8: 26404795- | PNMA2 | 0.008479148 | 2 | 0 |
| 26404795 | | | | |
| chr1: 174500555- | RFWD2.RP11- | 0.008467635 | 2 | 0 |
| 174543675 | 318C24.3 | | | |
| chr12: 18801189- | CAPZA3 | 0.00287034 | 3 | 1 |
| 18821605 | | | | |

To address the potential biological role of some of the other genes we identified all of which included CNVs that were either associated with or over-represented in schizophrenia, we performed Functional Annotation Clustering (FAC) using the DAVID Bioinformatics Database. We observed that deleted genes classified with GO term ionotropic glutamate receptor activity (p=5.8×10⁻⁴) and the Neuroactive Ligand-Receptor Interaction by Kegg pathway (p=5.5×10⁻³) had significant enrichment among these schizophrenia candidate genes, which have striking biological relevance to schizophrenia. Genes in the ionotropic glutamate receptor activity GO category include GRIK1 (glutamate receptor, ionotropic, kainate 1), GRIA4 (glutamate receptor, ionotrophic, ampa 4), GRIN3A (glutamate receptor, ionotropic, n-methyl-d-aspartate 3a), and GRIK5 (glutamate receptor, ionotropic). The twelve associated genes in the Neuroactive Ligand-Receptor Interaction pathway include TACR3 (tachykinin receptor 3), GRIK1 (glutamate receptor, ionotropic, kainate 1), FSHR (follicle stimulating hormone receptor), GRIA4 (glutamate receptor, ionotrophic, ampa 4), GRIN3A (glutamate receptor, ionotropic, n-methyl-d-aspartate 3a), GABRG2 (gamma-aminobutyric acid (gaba) a receptor, gamma 2), LEPR (leptin receptor, TRH thyrotropin-releasing hormone), GRIK5 (glutamate receptor, ionotropic, kainate 5, NTS neurotensin), GRM5 (glutamate receptor, metabotropic 5), and MC4R (melanocortin 4 receptor). These CNV containing genes have direct functional relevance to the development of schizophrenia. Several other genes are affected by the CNVs we have observed. The strength of the association signals suggests that these genes and potentially also their neighboring regions predispose to the schizophrenia phenotype.

In addition, we have identified 93 genes directly impacted by deletions that are overrepresented in the schizophrenia cases in comparison with the controls. None of those have been reported in the public domain in relation with schizophrenia and they are not listed in the reference database from Toronto, the Toronto Database of Genomic Variants. These genes are listed below:
CCL8, SHOC2, NTS, GRIK5, GRB14, RGS21, AF086288, SIAT6, ST3GAL3, ST3GalIII, AK055533, CACNA1S, CSRP1, DKFZp434B1231, HNTN1, LAD1, PHLDA3, PKP1, TMEM9, TNNI1, TNNT2, LIN9, C1orf131, AK094343, TACSTD1, LOC51057, CNNM4, AK024261, AK090954, RBM6, BC022563, DKFZp761B107, BC035172, OTUD4, FER, CHSY2, FARS2, LOC648232, AHI1, C7orf26, ZDHHC4, KIAA0744, TOX, ZFPM2, DCC1, DEPDC6, ENPP2, TAF2, C9orf68, C9orf123, DKFZp434O1230, ZEB1, CUL2, KIAA1279, AK056108, C10orf96, GFRA1, PNLIP, PNLIPRP1, PNLIPRP3, KIAA0652, FAM118B, FOXRED1, SRPR, TIRAP, KLRD1, MYO1A, SYT1, JIK, TAOK3, AK054970, AKAP11, AK125018, BC035119, BX247990, TCL1A, TCL1B, GALK2, DKFZp547H074, RNF111, EMP2, RUNDC2A, BC042382, CBLN2, OR7C2, SLC1A6, EHD2, BMP2, CHMP4B, BC043580, GRIK1, RUNX1, TTC3

An additional 193 genes are directly impacted by duplications that are overrepresented in the schizophrenia cases in comparison with the controls and not seen in Toronto Database of Genomic Variants. These genes are listed below:
AKT1, SIVA1, IL4R, NCLN, C20orf26, CRNKL1, FLJ31568, KIAA1978, MGC19604, RAVER1, LOC388595, Na+,SCN7A, CHPF, KIAA0657, MGC99813, CDK9, FPGS, ATP10C, AK127352, ABHD8, AK055623, ANKRD41, BST2, C19orf58, FAM125A, GTPBP3, MRPL34, PCIA1, PLVAP, TMEM16H, NLRC3, MGAT4C, BC044614, METRNL, MGC24975, TMEM146, CASKIN2, KIAA1139, TSEN54, CR592675, DEFB110, DEFB111, DEFB112, TFAP2B, TFAP2D, C7orf26, DAGLB, DAGLBETA, DC1, EIF2AK1, JTV1, KDELR2, MGC12966, RAC1, ZDHHC4, AX746719, DKFZp547O168, ZNF430, ZNF431, ZNF714, ZNF85, PCDH17, PCH68, DNMBP, TRPV2, VRL, CA11, D87947, DBP, FLJ36070, FUT2, IZUMO1, LOC126147, RASIP1, RPL18, SPACA4, SPHK2, SPHK2, SSTR4, AK128554, AK129550, AK131520, BC034980, BC071811, CADM4, DKFZp564H1322, FLJ12886, IRGC, IRGQ, KCNN4, LYPD3, LYPD5, PHLDB3, PLAUR, UNQ491, XRCC1, ZNF428, ZNF575, ZNF576, HSPA9, CSMD2, ZSCAN20, PTGFR, GBP2, GBP7, D28435, SNRPE, ZC3H11A, USH2A, RGS7, MTX2, NCL, CCDC14, DKFZp313E037, DKFZp434B1222, MLCK, MYLK, ROPN1, BC035722, BC036345, C4orf36, SLC10A6, ANK2, LOC340156, AK056211, GPX5, GPX6, ZNF452, gpx5, ENPP4, ENPP5, ASCC3, CPVL, DYNC1I1, UNC5D, ZFPM2, C8orf78, TMEM65, BC009730, BC041044, CR606996, IL33, KIAA1432, KIAA1815, KIAA2026, MLANA, NIRF, PDCD1LG2, RANBP6, UHRF2, ACER2, ASAH3L, SLC24A2, AQP3, NOL6, BC040625, DIRAS2, DQ584857, DQ585001, DQ596414, BSPRY, WDR31, SLC29A3, C10orf56, PPIF, BC019904, CD81, TRPM5, TSSC4, SLC39A13, SPI1, AL832007, BC041984, FZD4, PRSS23, TMEM135, ZSIG13, CRADD, BRMS1L, GARNL1, ABAT, TMEM186, KIAA1703, FLJ14959, EIF3S12, ASXH1, ASXL1, C20orf112, COMMD7, F1133706, LOC149950, BX648826

Taken together, these results suggest that the genetic landscape in the pathogenesis of schizophrenia involves both common and rare CNVs, that associate with the schizophrenia phenotypes, where the rare CNVs are highly heterogeneous and in many instances unique to the individual families and cluster on genes that are involved with neuronal signaling and development.

### REFERENCE FOR EXAMPLE I

1. Wang, K. et al. PennCNV: an integrated hidden Markov model designed for high-resolution copy number variation detection in whole-genome SNP genotyping data. Genome Res 17, 1665-1674 (2007).
2. Walsh, T., McClellan, J., McCarthy S. et al. Rare Structural Variants Disrupt Multiple Genes in Neurodevelopmental Pathways in Schizophrenia. Science 320(5875 539-543 (2008).
3. Kinkead B, Nemeroff C B. Neurotensin, schizophrenia, and antipsychotic drug action. Int Rev Neurobiol. 59 327-49 (2004).
4. A Gray and B L Roth, “The pipeline and future of drug development in schizophrenia” Molecular Psychiatry. 12 (10) 904-922. (2007).
5. N A Sachs et al. A frameshift mutation in Disrupted in Schizophrenia 1 in an American family with schizophrenia and schizoaffective disorder. Molecular Psychiatry 10, 758-764 (2005).
6. Cantor R. M. and Daniel H. Geschwind. Schizophrenia: Genome, Interrupted. Neuron, Volume 58, Issue 2, 165-167, (2008).
7. Makino .C. et al. .Positive association of the AMPA receptor subunit GluR4 gene (GRIA4) haplotype with schizophrenia: Linkage disequilibrium mapping using SNPs evenly distributed across the gene region American Journal of Medical Genetics Part B: Neuropsychiatric Genetics 116B Issue 1, Pages 17-22 (2002).
8. Hashimoto R. et al. A missense polymorphism (H204R) of a Rho GTPase-activating protein, the chimerin 2 gene, is associated with schizophrenia in men. Schizophrenia Research, 73, Issue 2-3 383-385 (2005).
9. Eastwood. S L et al. The axonal chemorepellant semaphorin 3A is increased in the cerebellum in schizophrenia and may contribute to its synaptic pathology Molecular Psychiatry 8, 148-155(2003).
10. G Dennis Jr et al. DAVID: Database for Annotation, Visualization, and Integrated Discovery. Genome Biology. 4(9), (2003).
11. Huang da W. et al. The DAVID Gene Functional Classification Tool: a novel biological module-centric algorithm to functionally analyze large gene lists. Genome Biology. 8(9), (2007).

### EXAMPLE II

### Strong Synaptic Transmission Impact by Copy Number Variations in Schizophrenia

Schizophrenia is a late adolescence onset psychiatric disease of unclear etiology characterized by both positive and negative symptoms as well as cognitive deficits. To identify copy number variations (CNVs) increasing risk of schizophrenia, we performed a whole-genome CNV analysis on a cohort of 977 schizophrenia cases and 2,000 healthy adults of European ancestry who were genotyped with 1.7 million probes. Positive findings were evaluated in an independent cohort of 580 schizophrenia cases and 1,485 controls. The Gene Ontology synaptic transmission family was notably enriched in the cases (P=1.5×10⁻⁷). Among those, CACNA1B and DOC2A, both calcium signaling genes responsible for neuronal excitation, were deleted in 16 cases (P=4.56×10⁻⁴) and duplicated in 10 cases (P=6.13×10⁻⁵), respectively. In addition, RET and RIT2, both ras related genes important for neural crest development, were significantly impacted by CNV. RET deletion was exclusive to 7 cases (P=5.11×10⁻³) and RIT2 deletions were overrepresented common variant CNVs in the schizophrenia cases (P=5.05×10⁻²). Our results indicate that variations involving synaptic transmission may contribute to the genetic susceptibility of schizophrenia.

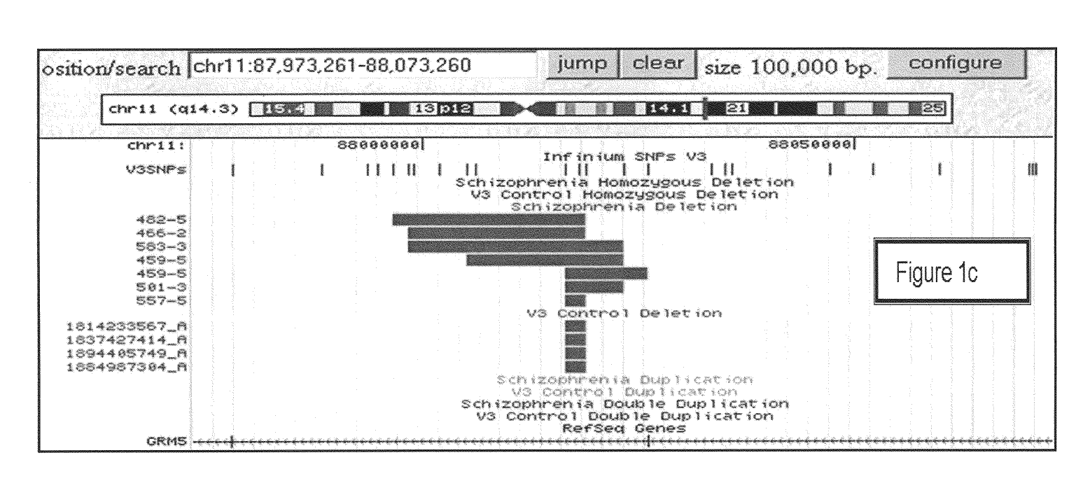

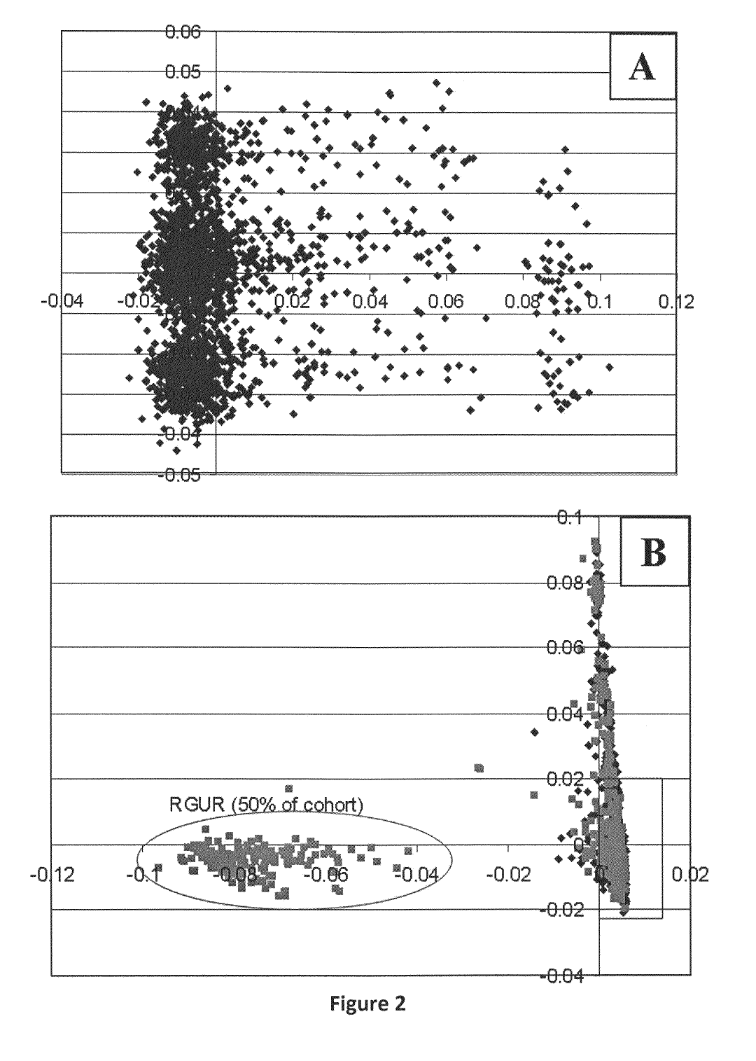

Various array technologies have been used to identify CNVs in healthy subjects, including aCGH, Affymetrix GeneChip and Illumina BeadChip. These studies have revealed significant common variation in the general healthy population¹⁹. Various algorithms are also being used to call CNVs most of which utilize the Hidden Markov Model as implemented in PennCNV²⁰. Clustering of all Affymetrix data in one run with Affymetrix Power Tools (APT), which implements BirdSeed, is essential to minimize stratification resulting from clustering bias. Indeed, the genotypes provided by dbGap in matrix format had significant clustering bias between three apparent runs of APT on sample subsets based on Eigenstrat analysis. We ran APT with all Affymetrix 6.0 samples in one run yielding a typical result showing few African and Asian admixed samples without the three modes from clustering bias (See FIG. 2). Thus, an informatic batch effect was resolved and a less addressable processing variation was not detected. Variables such as sample collection site and five character processing codes showed minimal bias. Differential CNV detection bias introduced by array batch effects is certainly a concern but given large case and control sets typed at both Stanford and CHOP, should not vary significantly between cases and controls.

The following materials and methods are provided to facilitate the practice of Example II.

### Affymetrix 6.0 Assay for CNV Discovery

High-throughput, genome-wide SNP and CN genotyping was performed, using the Affymetrix 6.0 technology, at the Center for Applied Genomics at CHOP. dbGaP samples were genotyped on the same platform at the Stanford University. The genotype data content together with the intensity data provided by the SNP probes on the genotyping array provides high confidence for CNV calls. Importantly, the simultaneous analysis of intensity data and genotype data in the same experimental setting establishes a highly accurate definition for normal diploid states and any deviation thereof. To call CNVs, we used the PennCNV-Affy algorithm, which combines multiple sources of information, including Log R Ratio (LRR) and B Allele Frequency (BAF) at each SNP marker, along with SNP spacing and population frequency of the B allele to generate CNV calls.

### CNV Quality Control

We calculated Quality Control (QC) measures on our Affymetrix 6.0 and HumanHap550 GWAS data based on statistical distributions to exclude poor quality DNA samples and false positive CNVs. The first threshold is the percentage of attempted SNPs which were successfully genotyped. Only samples with call rate >96% were included. The genome wide intensity signal must have as little noise as possible. Only samples with the standard deviation (SD) of normalized intensity (LRR) <0.35 were included. All samples must have Caucasian ethnicity based on hierarchical clustering of AIMs genotypes and all other samples were excluded. Wave artifacts roughly correlating with GC content resulting from hybridization bias of low full length DNA quantity are known to interfere with accurate inference of copy number variations (35). Only samples where the GC-wave factor (GCWF) of LRR was between −0.02<X<0.02 were accepted. If the count of CNV calls made by PennCNV exceeds 80 (FIG. 2), the DNA quality is usually poor. Thus, only samples with CNV call count <80 were included. Any duplicate samples (such as monozygotic twins) had one sample excluded.

### Statistical Analysis of CNVs

CNV frequency between cases and controls was evaluated at each SNP using Fisher's exact test. We only considered loci that were significant between cases and controls (p<0.05) where cases in the MGS/Gur discovery cohort had the same variation, replicated in MGS/Gur or were not observed in any of the control subjects, and validated with an independent method. We report statistical local minimums to narrow the association in reference to a region of nominal significance including SNPs residing within 1 Mb of each other. Resulting significant CNVRs were excluded if they met any of the following criteria: i) residing on telomere or centromere proximal cytobands; ii) arising in a “peninsula” of common CNV arising from variation in boundary truncation of CNV calling; iii) genomic regions with extremes in GC content which produces hybridization bias; or iv) samples contributing to multiple CNVRs. We used DAVID (Database for Annotation, Visualization, and Integrated Discovery) (36) to assess the significance of functional annotation clustering of independently associated CNV results into functional categories. To adjust for number of tests performed, we made correction of 21 deletion and 5 duplication CNVRs, based on significance in the discovery cohort.

### CNV Validation by Quantitative PCR

Universal Probe Library (UPL; Roche, Indianapolis, Ind.) probes were selected using the ProbeFinder v2.41 software (Roche, Indianapolis, Ind.). Quantitative PCR was performed on an ABI 7500 Real Time PCR Instrument or on an ABI Prism™ 7900HT Sequence Detection System (Applied Biosystems, Foster City, Calif.). Each sample was analyzed in quadruplicate either in 25 μl reaction mixture (250 nM probe, 900 nM each primer, Fast Start TaqMan Probe Master from Roche, and 10 ng genomic DNA) or in 10 μl reaction mixture (100 nM probe, 200 nM each primer, 1× Platinum Quantitative PCR SuperMix-Uracil-DNA-Glycosylase (UDG) with ROX from Invitrogen, and 25 ng genomic DNA). The values were evaluated using Sequence Detection Software v2.2.1 (Applied Biosystems, CA). Data analysis was further performed using either the ΔΔC_{T}, method or qBase. Reference genes, chosen from COBL, GUSB, and SNCA, were included based on the minimal coefficient of variation and then data were normalized by setting a normal control to a value of 1.

### PennCNV-Affy

The CNV calling on Affymetrix 6.0 platform used a highly similar algorithm as those used in the Illumina arrays, but the signal pre-processing steps differ. Unlike the Illumina platform, where normalized signal intensities (Log R Ratio and B Allele Frequency) can be exported directly from the BeadStudio software, these signal intensity measures in the Affymetrix platform need to be calculated from the collection of genotyped samples. We used the Affymetrix Power Tools on the world wide web at affymetrix.com/support/developer/powertools/changelog/index.html) to perform data normalization and signal extraction from raw CEL files generated in genotyping experiments. The “median smoothing” and “quantile normalization” options were used in the Affymetrix Power Tools. The expr.genotype=true option was also used to specify allele-specific signal extraction. This step uses a self-normalization algorithm that requires information contained within all the genotyped samples. The Affymetrix Power Tools software was also used for genotype calling, and a “confidence score” is assigned to each genotype call. For each SNP marker, we then relied on the allele-specific signal intensity for the AA, AB and BB genotypes on all genotyped samples to construct three canonical genotype clusters, similar to the Illumina clustering generation approach. Genotype calls with confidence score less than 0.1 were not used in the construction of canonical genotype clusters. Once the canonical genotype clusters have been constructed, we can then transform the signal intensity values for each SNP to Log R Ratio (LRR) and B Allele Frequency (BAF) values.

The Affymetrix arrays contain non-polymorphic (NP) markers to provide better genome coverage than SNP markers only. These markers can be handled in a fashion similar to SNPs for copy number inference, but there are some differences. First, the R-value is calculated as the signal intensity of the NP marker rather than the sum of two alleles. The expected R value for each NP marker is calculated as the median signal intensity values for all genotyped samples at this marker. Also, the BAF values cannot be derived for NP markers. Consequently, they are not used in the likelihood calculation. Finally, due to the use of fewer probes, the variance of LRR values for NP markers may be different than SNP markers. Therefore, the likelihood model parameters for LRR are different between NP markers and SNP markers.

### Illumina Infinium Assay for CNV Calling

The genotype data content together with the intensity data provided by the genotyping array provides high confidence for CNV calls. The array platform used in this study provides a highly robust and reproducible SNP clustering due to the random placement of SNP specific beads with approximately 18-fold redundancy for each SNP. Using a SNP array provides allele frequency data which can be analyzed and more closely quality controlled for redundancy and high performance when compared to public databases. This establishes a more robust definition for normal diploid states than can be provided by intensity alone. The genotype clustering establishes the probe performance at each locus for the expected heterozygous genotype state. Based on the hybridization efficiency, this may tend more to the DNP tagged Red range or the Biotin tagged Green range for any given locus. The normalization performed to calculate B allele frequency (BAF) from theta adjusts the SNP specific range to a 0.5 expected value. This creates more continuous data since the heterozygous state is properly modeled based on extensive genotyping. Another key technical strength of our study is that the same array was typed at the same genotyping facility at the same time with the same cluster file for cases and controls. The data analysis is also standardized as described in the methods and CNVs are called with the same version of PennCNV.

### CNV Filtering Steps

Multiple CNV filtering steps have been performed as part of the analysis. First, it is important to note that of the 1,736,438 markers (848,415 SNP and 888,023 CN) with chromosome annotation, non-complete genotyping failure, 3 genotype states observed, and normal theta patterns on the Affymetrix 6.0 array, 33,797 (10,687 SNPs and 23,110 CN) (1.95%) showed deletion and 44,023 (16,618 SNPs and 27,405 CN) (2.54%) showed duplication in at least two or more unrelated cases in the MGS/CHOP discovery cohort (frequency ≧0.205%). The threshold of two cases is selected because it is the minimal case frequency to provide certainty that the calls are reliable in a given region. We find this upfront exclusion to be very similar to the inclusion threshold of 1% Minor Allele Frequency in GWA SNP genotype studies. This drastically cuts down on the number of test preformed to correct for genome wide testing.

Secondly, all CNVs were called simultaneously in both cases and controls and classified into CNVRs as defined in Example II. A total of 70 deletion and 50 duplication CNVRs were identified. Thirdly, to search for novel CNVs, we first filtered out all CNVRs that were not nominally significantly overrepresented in the CHOP cases (P<0.05) and carefully reviewed the raw data (BAF and LRR) for accurate CNV calling and statistical significance as described in Methods. This left us with 20 deletion and 5 duplication CNVRs that we subsequently divided into two categories: i) CNVs present in cases only and absent in controls: N=5 deletions and 2 duplications. Based on the inclusion significance criteria, there were at least 2 or more cases per individual CNV. ii) CNVs nominally significantly overrepresented in the cases: N=15 deletions and 3 duplications.

This dataset (i) and (ii) therefore defines the CNVRs from the discovery cohort that we used to test for novel schizophrenia CNVs. We next attempted replication of these CNVRs in the independent case-control dataset (MGS/CHOP). Seven deletion and one duplication CNVRs survived our replication criteria (P value <0.05 following adjustment for the number of tests performed—or they were absent in the independent control set) and were subsequently experimentally validated with two independent methods (QPCR and Illumina Human Hap550 Beadchip). These results are shown in Table 4.

TABLE 4
CNVRs Statistically Overrepresented in Schizophrenia Cases and Replicated in an Independent Case-Control Cohort

| | | | | | Con- | | Con- | | | | | |
|---|---|---|---|---|---|---|---|---|---|---|---|---|
| | | | | Cases | trols | Cases | trols | | Distance | | Repli- | |
| | | | | Dis- | Dis- | Repli- | Repli- | | From | | cation | Ca- |
| CNVR | Probes | P Value | OR | covery | covery | cation | cation | Gene | Gene | Type | ISC | nary |
| chr16: 68743639- | 9 | 3.55 × 10⁻⁶ | 4.008 | 19 | 10 | 11 | 9 | PDPR | 0 | Del | 6:1 ISC | N |
| 68770545 | | | | | | | | | | | p = 0.13 | |
| chr22: 17404806- | 1529 | 7.73 × 10⁻⁶ | NA | 8 | 0 | 2 | 0 | 75 Genes | 0 | Del | 11:0 ISC | Y |
| 19941349 | | | | | | | | COMT | | | p = 0.001 | |
| chr16: 29425212- | 217 | 6.13 × 10⁻⁵ | 22.52 | 5 | 0 | 5 | 1 | 52 Genes | 0 | Dup | 6:3 ISC | Y |
| 30134444 | | | | | | | | QPRT, DOC | | | p = 0.51 | |
| | | | | | | | | 2A, TBX6 | | | | |
| chr9: 140145139- | 7 | 4.56 × 10⁻⁴ | 4.513 | 12 | 4 | 4 | 4 | CACNA1 B | 8.69 kb | Del | — | Y↑ |
| 140152969 | | | | | | | | | | | | |
| chr10: 42932615- | 17 | 5.11 × 10⁻³ | 7.865 | 5 | 0 | 2 | 2 | RET | 0 | Del | — | N |
| 42934354 | | | | | | | | | | | | |
| chr3: 4063809- | 30 | 3.10 × 10⁻² | 1.959 | 14 | 13 | 6 | 10 | SUMF1 | 0 | Del | — | N |
| 4074877 | | | | | | | | | | | | |
| chr4: 9881886- | 11 | 3.71 × 10⁻² | 2.810 | 6 | 2 | 4 | 6 | WDR1 | 154 kb | Del | — | Y |
| 9884092 | | | | | | | | | | | | |
| chr18: 38310567- | 25 | 5.05 × 10⁻² | 1.224 | 115 | 163 | 46 | 137 | RIT2, | 265 kb, | Del | — | Y |
| 38311765 | | | | | | | | PIK3C3 | 395 kb | | | |

Significant CNVRs based on a combined discovery and replication cohort of 1,557 schizophrenia cases and 3,485 healthy controls of European ancestry. Replication ISC- samples from different sample sources must have reasonable contributing frequency. Canary- A CNV calling algorithm run on the sample set in addition to PennCNV-Affy to establish independent calling positive replication (Y) or lack of replication (N). ↑ indicates more samples with Canary calls. Del: Deletion Dup: Duplication. CNVRs that survive multiple testing with Bonferroni adjustment in the discovery phase (P < 0.05 following correction for 20 tests in case of deletion and 5 in case of duplications), survived replication and experimental validation are listed in bold. The CNVR is the CNV region shared significant region between cases. Probes gives the number of SNP and CN probes present on the Affymetrix 6.0 array in the given CNVR from which signal was indicative of a CNV. The P-value is based on a Fisher's exact test of the combined sample. The count of samples in each subgroup of cases and controls in discovery and replication is provided. The nearest gene and proximal distance is provided for potential functional impact and a means to compare other sample sets which may finds CNVs in the region. The Replication ISC column shows the frequency of cases:controls in the International Schizophrenia Consortium CNV calls of 3,391 cases and 3,181 controls. Canary column shows if the analysis of the Log2 ratio of intensity through the Canary CNV calling algorithm replicates the CNV call from PennCNV-Affy. Key functional genes are provided for brevity. The gene count for the two largest CNVs includes hypothetical genes.

In Table 4, CNVRs that survive multiple testing with Bonferroni adjustment in the discovery phase (P<0.05 following correction for 20 tests in case of deletion and 5 in case of duplications), survived replication and experimental validation are listed in bold. CNVRs significant in the discovery phase but not in the replication phase are listed in Table 5.

TABLE 5
CNVs Statistically Overrepresented in Schizophrenia Cases and Not Replicated in an Independent Cohort

| | | | | | Con- | | Con- | | | | | |
|---|---|---|---|---|---|---|---|---|---|---|---|---|
| | | | | Cases | trols | Cases | trols | | Distance | | | |
| | | | | Dis- | Dis- | Repli- | Repli- | | From | | Repli- | Ca- |
| CNVR | Probes | P Value | OR | covery | covery | cation | cation | Gene | Gene | Type | cation | nary |
| chr7: 32177451- | 198 | 2.94 × 10⁻² | NA | 3 | 0 | 0 | 0 | PDE1C | 0 | Dup | | N |
| 32392975 | | | | | | | | | | | | |
| chr3: 61803641- | 9 | 3.42 × 10⁻² | 8.9736 | 4 | 0 | 0 | 1 | PTPRG | 0 | Del | | N |
| 61811383 | | | | | | | | | | | | |
| chr4: 135276704- | 21 | 4.76 × 10⁻² | 2.4966 | 7 | 2 | 3 | 7 | PABPC4L | 0 | Del | | N |
| 135408238 | | | | | | | | | | | | |
| chr5: 2097129- | 17 | 6.37 × 10⁻² | 2.2471 | 9 | 7 | 2 | 4 | IRX4 | 161 kb | Del | | N |
| 2111366 | | | | | | | | | | | | |
| chr12: 60558836- | 10 | 6.37 × 10⁻² | 2.2471 | 11 | 7 | 0 | 4 | FAM19A2 | 0 | Del | 2 RG | N |
| 60563972 | | | | | | | | | | | | |
| chr6: 57268143- | 13 | 7.76 × 10⁻² | 4.4855 | 4 | 0 | 0 | 2 | PRIM2A, | 17.9 kb | Del | | N |
| 57272458 | | | | | | | | RAB23 | 73.1 kb | | | |
| chr5: 52702915- | 12 | 1.87 × 10⁻¹ | 1.9947 | 7 | 5 | 1 | 4 | FST | 109 kb | Del | | N |
| 52718131 | | | | | | | | | | | | |
| chr19: 426716- | 5 | 2.11 × 10⁻¹ | 1.7947 | 3 | 1 | 5 | 9 | SHC2 | 14.7 kb | Dup | | N |
| 434473 | | | | | | | | | | | | |
| chr6: 16499554- | 20 | 2.40 × 10⁻¹ | 1.9221 | 6 | 2 | 0 | 5 | ATXN1 | 0 | Dup | 1 RG | N |
| 16508717 | | | | | | | | | | | | |
| chr15: 99980078- | 36 | 3.01 × 10⁻¹ | 2.2423 | 5 | 2 | 0 | 3 | TM2D3, | 0 | Dup | | N |
| 100033288 | | | | | | | | TARSL2, | | | | |
| | | | | | | | | TM2D3 | | | | |
| chr15: 32717247- | 50 | 3.21 × 10⁻¹ | 1.3356 | 15 | 15 | 7 | 22 | GJD2 | 0 | Del | | N |
| 32765105 | | | | | | | | | | | | |
| chr7: 142941348- | 10 | 3.21 × 10⁻¹ | 1.6311 | 8 | 3 | 0 | 8 | AL833583 | 10.7 kb | Del | | N |
| 142963649 | | | | | | | | | | | | |
| chr4: 114573691- | 27 | 5.10 × 10⁻¹ | 1.4935 | 4 | 1 | 0 | 5 | CAMK2D | 11.7 kb | Del | | N |
| 114581335 | | | | | | | | | | | | |
| chr4: 162417655- | 12 | 5.10 × 10⁻¹ | 1.4935 | 4 | 0 | 0 | 6 | FSTL5, | 99.9 kb | Del | 1 RG | Y |
| 162424561 | | | | | | | | RAPGEF2 | 1.92 Mb | | | |
| chr6: 162740476- | 2 | 5.32 × 10⁻¹ | 1.6007 | 5 | 4 | 0 | 3 | PARK2 | 0 | Del | | N |
| 162741040 | | | | | | | | | | | | |
| chr1: 92014319- | 10 | 5.56 × 10⁻¹ | 1.4002 | 5 | 3 | 0 | 5 | TGFBR3 | 0 | Del | | N |
| 92021028 | | | | | | | | | | | | |
| chr12: 69158942- | 9 | 1 | 0.9322 | 8 | 6 | 2 | 18 | PTPRB, | 32.6 kb, | Del | | N |
| 69164294 | | | | | | | | KCNMB4 | 47.7 kb | | | |

Conversely, only one CNV locus overrepresented in controls reached nominal significance. Therefore CNVs overrepresented in cases exceeded our null expectations. Given the diploid state of the vast majority of the genome, the existence of CNVs protective against the development of schizophrenia seems unlikely.

### Results

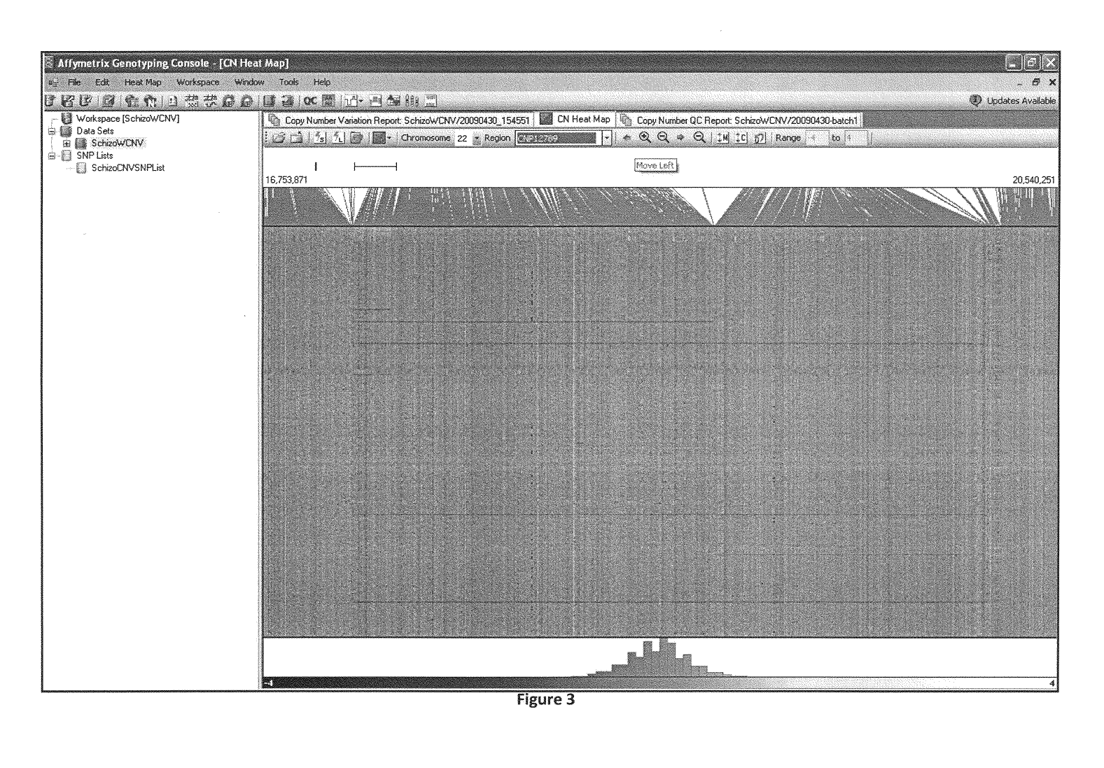

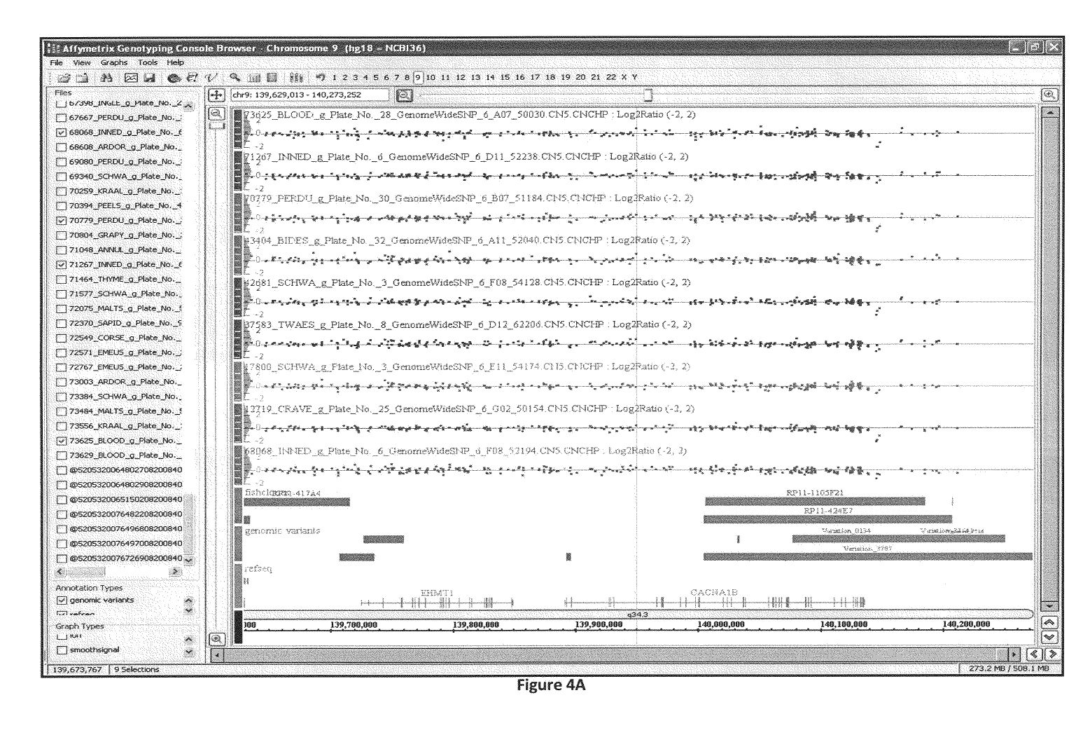

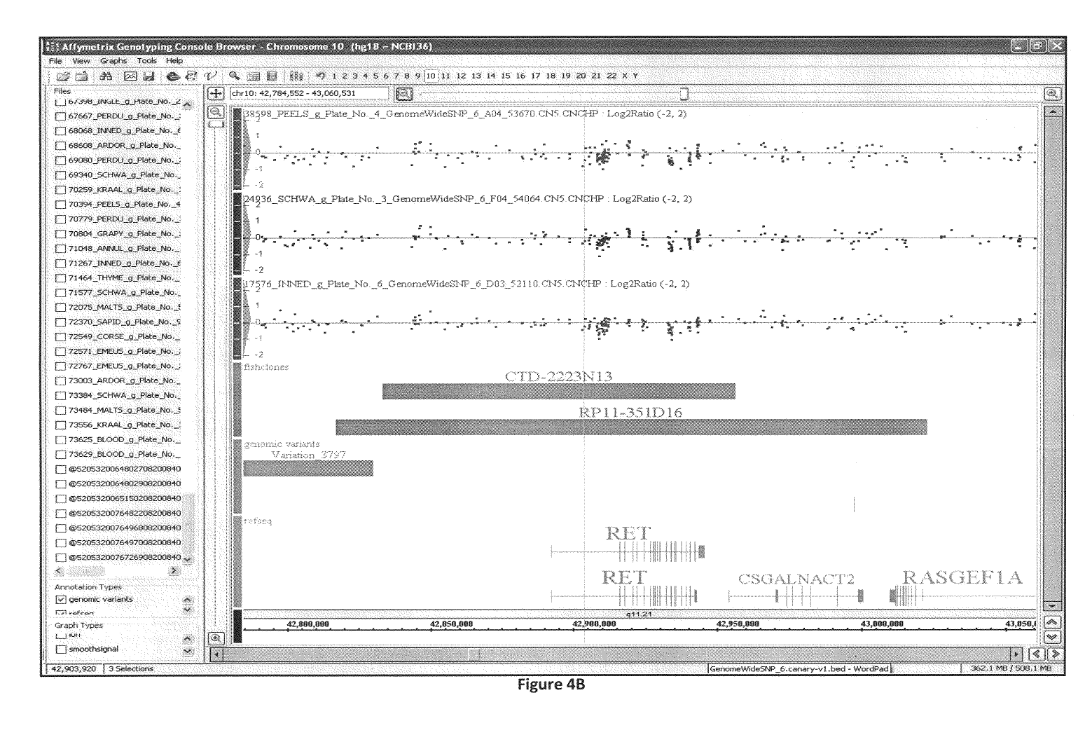

The Affymetrix 6.0 provided 848,415 SNP markers and 888,023 CN markers that were analyzed to construct canonical clustering positions using the PennCNV-Affy workflow, which normalizes the Cartesian coordinates provided by Affymetrix. PennCNV-Affy utilizes called genotypes and normalizes intensity from Affymetrix Power Tools (APT) to create reference cluster positions in polar coordinates to compute relative differences in the signal from each sample in the form of B-allele frequency (BAF) and Log R Ratio (LRR). BAF, LRR, population BAF, inter-probe distance, and HMM model files were then analyzed by PennCNV to make CNV calls for each sample. We observed the same CNV call based on the Canary component of Birdsuite for many CNVs. We reviewed the Log 2 Ratio values in visualization tools Affymetrix Genotyping Console Heat Map (FIG. 3) and Browser (FIG. 4). However, PennCNV-Affy calls are preferred due to their use of Log R Ratio rather than Log 2 Ratio. The Log 2 Ratio is based on quantile normalization, the sum of signal intensity for A allele and B allele for each sample, the median across all samples, and for a given sample, divide A+B allele intensity by the median value and take the logarithm base 2. In contrast, the Log R Ratio is based on defined signal intensity clusters of AA, AB and BB genotypes across a large group of samples. Given this expected intensity value, the observed A+B signal intensity data is divided by this expected value, and the logarithm taken. Although the number of CNVs called per individual by PennCNV-Affy may be lower than BirdSuite, this smaller CNV set has a lower false positive rate which is crucial.

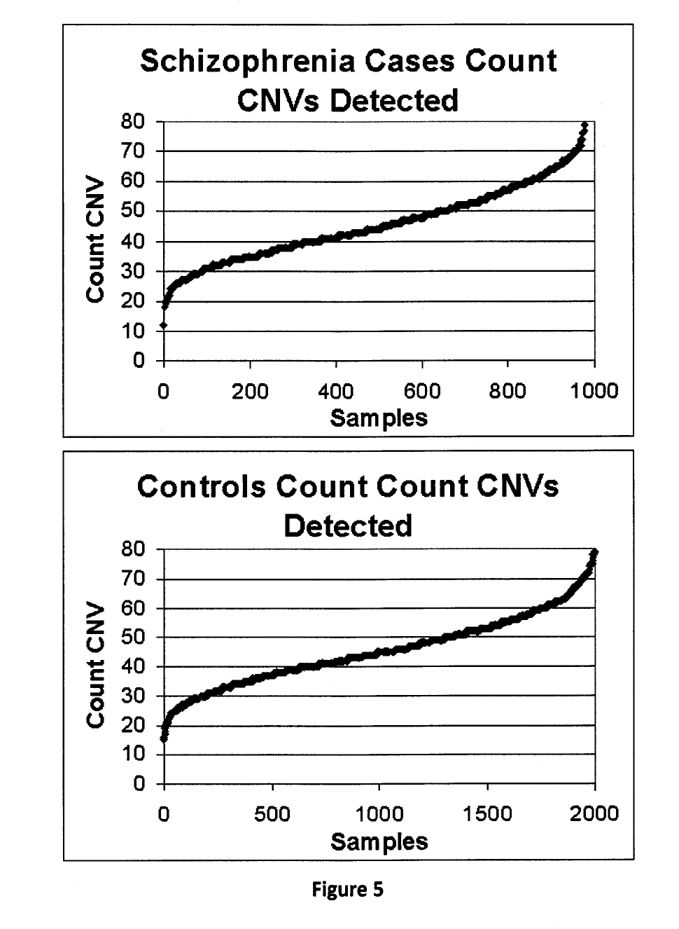

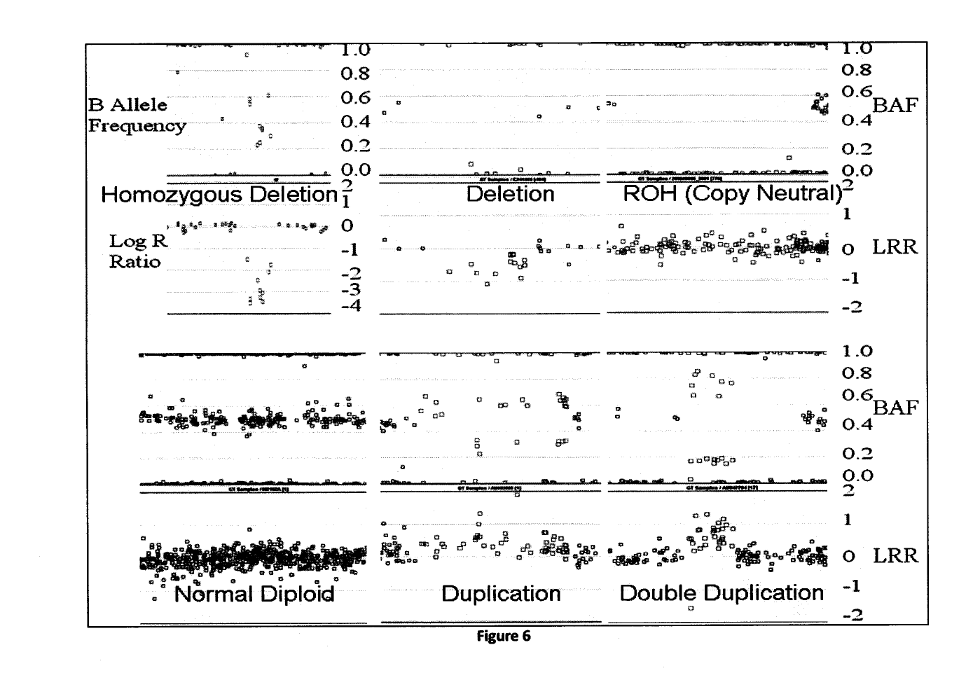

We analyzed a total of 1,557 case Affymetrix 6.0 samples that met strictly established data quality thresholds for copy number variation for the discovery phase of 977 cases and the replication phase of 580 cases. An average of 45.4 CNV calls was made for each individual using the PennCNV software. Each individual included had a CNV frequency between 1-80 CNV calls (FIG. 5). We called four different copy number states, including 9,059 homozygous deletions (copy number, or CN=0), 21,526 hemizygous deletions (CN=1), 9,750 duplications (CN=3), and 4,024 duplications (CN=4). FIG. 6 shows raw BAF and LRR and the resulting CNV call. The CNV calls spanned from 3 to 3,253 probes, with an average of 48 probes per CNV call, and their sizes ranged from 6 by to 8.1 Mb, with an average size of 88.4 kb. The CNV calls from the schizophrenia cases were compared with those from 3,485 healthy subjects. Control individuals examined also had CNV frequency ranging from 1-80 CNV calls per subject (FIG. 2). An average of 45.1 CNV calls were made for each control individual using the PennCNV software. Among them, we identified 29,257 homozygous deletions (CN=0), 70,052 hemizygous deletions (CN=1), 32,906 duplications (CN=3), and 14,217 duplications (CN=4). The CNV calls spanned from 3 to 9,258 probes, with an average of 48.6 probes per CNV call, and their sizes ranged from 4 bp to 12.7 Mb, with an average size of 87.9 kb.

In an attempt to replicate and better classify the reported abundance of rare CNVs in schizophrenia cases, we determined CNV case and control frequencies applying different CNV association conditions: 100+ kb CNV size, 100+ kb CNV size and not present in the Database of Genomic Variants (DGV), 10+ probe CNV size, 10+ probe CNV Size and not present in DGV, and samples with multiple novel genes impacted by CNVs. The 100 kb CNV size inclusion threshold excludes many CNVs that are informative and could impact many of the loci presented as novel to cases. For example, using the 100 kb threshold would have excluded 77% of the CNV calls in our discovery cohort. In contrast, CNVs called with 10 probes show a low false positive rate based on experimental validation of our studies and results in exclusion of only 6% of our called CNVs. When using a threshold for CNV calls sized 100 kb and larger, we replicated the 22q11.2 deletions robustly, and we detected CNV association to GRID1, CNTNAP2, DISC1, and NRXN1, as previously reported. However, upon further review, there were multiple smaller CNVs present in these regions in both the cases and controls, suggesting that large CNVs in these regions may be required for strong risk of schizophrenia.

We next carried out single SNP association analysis genome wide. We did not detect any loci that were genome-wide significant, however, we detected nominally significant association to several genes that are essential for brain development and function, including but not limited to ASTN2, CNTN5, and GRIK2 (P=2.29×10⁻⁶, 6.63×10⁻⁶, and 2.53×10⁻⁵, respectively; Table 6). As demonstrated in reports associating the genotypes in the MHC locus with schizophrenia¹⁷, such nominal significance may exist in the analysis of a large cohort but may replicate with other groups resulting in a genome-wide significance. Indeed many do not directly impact genes, but most likely impact the nearest proximal gene based on linkage disequilibrium. We provide these SNP genotype association results as highly suggestive loci based on statistical significance and functional relevance.

TABLE 6
GWA of SNP Genotypes from 1067 Schizophrenia Cases and 1304 Controls

| | | | | | | | | | Count |
|---|---|---|---|---|---|---|---|---|---|
| SNP | P-value | Chr | Position | Gene | A1 | F_A | F_U | Distance | SNPs |
| rs4697472 | 1.35 × 10⁻⁶ | 4 | 24307401 | SOD3 | 1 | 0.4675 | 0.3973 | 85734 | 10 |
| rs1587434 | 1.73 × 10⁻⁶ | 6 | 66672076 | EYS | 2 | 0.0519 | 0.02527 | 191994 | 4 |
| rs11789407 | 2.29 × 10⁻⁶ | 9 | 120399367 | ASTN2 | 1 | 0.5203 | 0.4512 | 595986 | 8 |
| rs1555543 | 4.46 × 10⁻⁶ | 1 | 96717385 | PTBLP, PTBP2 | 1 | 0.4545 | 0.3884 | 298064 | 4 |
| rs35648 | 5.95 × 10⁻⁶ | 10 | 80171865 | AF086162 | 1 | 0.1242 | 0.1714 | 61304 | 6 |
| rs2155907 | 6.63 × 10⁻⁶ | 11 | 97599883 | CNTN5 | 2 | 0.4035 | 0.3393 | 778692 | 5 |
| rs2271293 | 9.96 × 10⁻⁶ | 16 | 66459571 | NUTF2 | 1 | 0.1425 | 0.1006 | 0 | 2 |
| rs4981929 | 9.96 × 10⁻⁶ | 14 | 31442403 | NUBPL | 1 | 0.527 | 0.462 | 2358 | 11 |
| rs11713590 | 1.12 × 10⁻⁵ | 3 | 5706142 | EDEM1 | 1 | 0.4225 | 0.4865 | 459455 | 10 |
| rs12140791 | 1.85 × 10⁻⁵ | 1 | 160357908 | NOS1AP | 2 | 0.06232 | 0.03569 | 0 | 2 |
| rs12538910 | 1.92 × 10⁻⁵ | 7 | 57418107 | DQ578920 | 1 | 0.4243 | 0.3633 | 50874 | 5 |
| rs10499040 | 2.53 × 10⁻⁵ | 6 | 104889038 | GRIK2 | 2 | 0.1345 | 0.09555 | 2264387 | 3 |
| rs1357338 | 1.19 × 10⁻⁴ | 1 | 174197509 | RFWD2 | 1 | 0.0188 | 0.00652 | 0 | 2 |
| rs4509495 | 1.33 × 10⁻⁴ | X | 42018121 | CASK | 2 | 0.1495 | 0.2015 | 196216 | 4 |
| rs4813376 | 1.92 × 10⁻⁴ | 20 | 19799455 | RIN2 | 2 | 0.1856 | 0.1453 | 18744 | 2 |
| rs6560936 | 4.43 × 10⁻⁴ | 13 | 113964074 | RASA3 | 2 | 0.5009 | 0.4497 | 40707 | 4 |

The most significant SNP is reported with neighboring SNPs within 10 kb and significance ranging within a power of ten noted by Count SNPs column. F_A: Allele frequency affected F_U: Allele frequency unaffected. Genes associated with brain development and function are listed in bold.

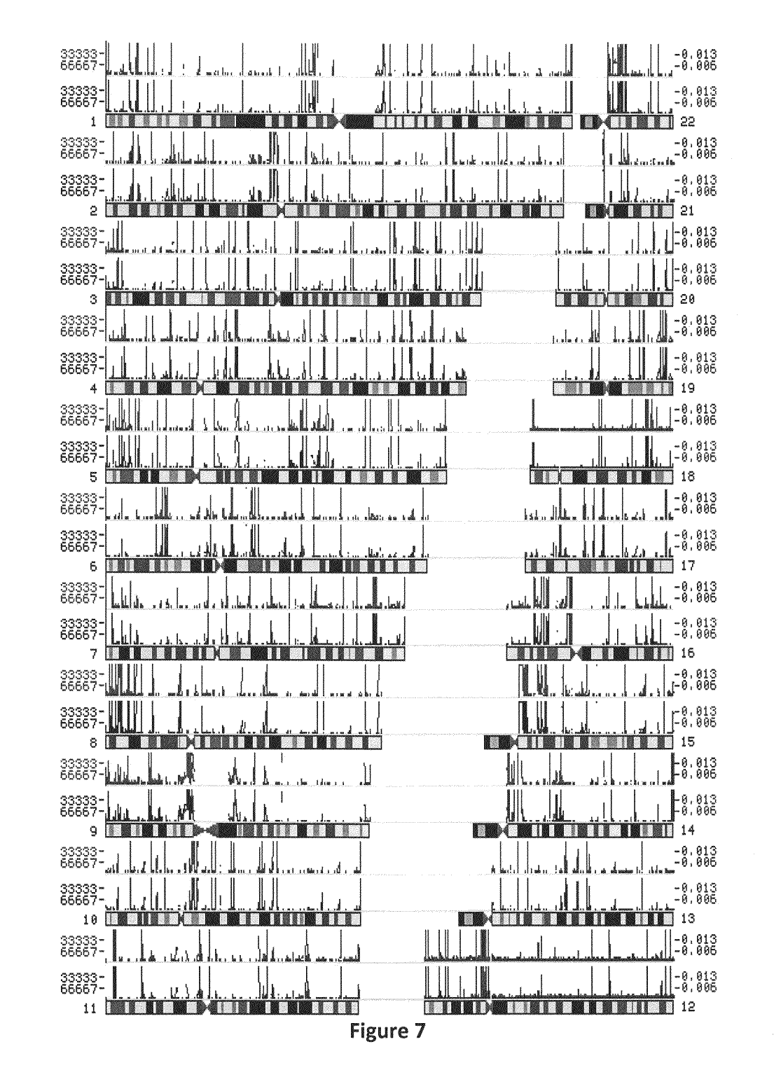

To identify novel CNV loci potentially contributing to schizophrenia, we applied a segment-based scoring approach that scans the genome for consecutive probes with more frequent copy number changes in cases compared to controls. See FIG. 7. The genomic span for these consecutive probes forms common copy number variation regions, or CNVRs. In the discovery cohort of 977 schizophrenia cases and 2,000 healthy subjects, we identified CNVRs that had significantly higher frequency in cases versus controls (Table 2 and Table 5 based on those that were also overrepresented in the replication cohort and those that failed replication, respectively). To assess the reliability of our CNV detection method, we experimentally validated all the significant CNVRs using two additional methods, Illumina Human Hap550 Beadchip and quantitative PCR (qPCR), which is widely used for independent validation of CNVs (Table 7). We examined CNV frequency of 4,000 healthy controls typed on the Illumina 550 array recruited by the Center for Applied Genomics at CHOP and we established CNV frequency in those samples close to that observed in controls typed on Affymetrix 6.0. Some regions had only one SNP represented on the Illumina array where Affymetrix had CN probe coverage, but samples showing deviations of the clustering of these SNPs allowed for CNV calls to be made. We validated all significant schizophrenia associated CNVs detected by the Illumina 550 chip with qPCR for two-tiered validation. Thus, we applied experimental validation on all the CNVRs to ensure positive confirmation of all final results reported. The false negative rate may be substantial based on conservative quality thresholds, but is not expected to be significantly different between case and control cohorts.

TABLE 7
Independent Validation of CNVRs with qPCR and Illumina Human Hap550 BeadChip

| | | | Relative | | | | Illumina |
|---|---|---|---|---|---|---|---|
| | CNV | | Gene | Standard | Illumina Chip | Tag SNP | Log R |
| CNVR | Type | Sample ID | Dosage | Error | ID | ID | Ratio |
| chr22: 17404806-19941349 | Del | 1222439226 | 0.524 | 0.035 | 4290041416_21 | rs1934895 | −1.052 |
| chr22: 17404806-19941349 | Del | 9626794429 | 0.521 | 0.011 | 4276098785_11 | rs1934895 | −0.996 |
| chr22: 17404806-19941349 | Del | 04C28087A* | 1.000 | 0.173 | 4562262038_21 | rs1934895 | −0.018 |
| chr22: 17404806-19941349 | Del | 04C28139A* | 1.029 | 0.122 | 4562369091_21 | rs1934895 | −0.120 |
| chr16: 29425212-30134444 | Dup | 7873015771 | 1.461 | 0.089 | 4079019681_A | rs4563056 | 0.498 |
| chr16: 29425212-30134444 | Dup | 8623080628 | 1.489 | 0.007 | 1582065333_A | rs4563056 | 0.595 |
| chr16: 29425212-30134444 | Dup | 9163054078 | 1.508 | 0.096 | 1846673715_A | rs4563056 | 0.369 |
| chr16: 29425212-30134444 | Dup | 04C28087A* | 1.000 | 0.023 | 4562262038_21 | rs4563056 | −0.063 |
| chr16: 29425212-30134444 | Dup | 04C28139A* | 0.975 | 0.027 | 4562369091_21 | rs4563056 | −0.221 |
| chr16: 68743639-68770545 | Del | 151169809 | 0.548 | 0.034 | 1587851079_A | rs17028422 | −0.135 |
| chr16: 68743639-68770545 | Del | 04C28087A* | 1.000 | 0.031 | 4562262038_21 | rs2287983 | −0.017 |
| chr16: 68743639-68770545 | Del | 04C28139A* | 0.954 | 0.017 | 4562369091_21 | rs2287983 | −0.059 |
| chr9: 140145139-140152969 | Del | 1475148472 | 0.507 | 0.246 | 4147907270_B | rs11137379 | −1.765 |
| chr9: 140145139-140152969 | Del | 3005849912 | 0.473 | 0.008 | 4068230324_B | rs11137379 | −2.270 |
| chr9: 140145139-140152969 | Del | 4311028436 | 0.475 | 0.029 | 4276098403_12 | rs11137379 | −2.711 |
| chr9: 140145139-140152969 | Del | 5678778794 | 0.545 | 0.128 | 1846673296_A | rs11137379 | −2.025 |
| chr9: 140145139-140152969 | Del | 6711973667 | 0.428 | 0.154 | 1796039438_A | rs11137379 | −1.951 |
| chr9: 140145139-140152969 | Del | 8934645510 | 0.432 | 0.023 | 4276098713_22 | rs11137379 | −2.440 |
| chr9: 140145139-140152969 | Del | 9140263548 | 0.474 | 0.020 | 4276098270_12 | rs11137379 | −2.804 |
| chr9: 140145139-140152969 | Del | 04C28087A* | 1.000 | 0.036 | 4562262038_21 | rs11137379 | −0.003 |
| chr9: 140145139-140152969 | Del | 04C28139A* | 1.035 | 0.091 | 4562369091_21 | rs11137379 | −0.136 |
| chr10: 42932615-42934354 | Del | 300030062 | 0.617 | 0.016 | 4276098188_12 | rs715106 | −0.175 |
| chr10: 42932615-42934354 | Del | 1207317307 | 0.527 | 0.041 | 4523255137_11 | rs715106 | −0.204 |
| chr10: 42932615-42934354 | Del | 1299194495 | 0.455 | 0.126 | 4506261167_11 | rs715106 | −0.161 |
| chr10: 42932615-42934354 | Del | 5442260823 | 0.488 | 0.168 | 4562297116_21 | rs715106 | −0.174 |
| chr10: 42932615-42934354 | Del | 9508038552 | 0.375 | 0.009 | 4157398294_A | rs715106 | −0.460 |
| chr10: 42932615-42934354 | Del | 04C28087A* | 1.000 | 0.026 | 4562262038_21 | rs715106 | −0.003 |
| chr10: 42932615-42934354 | Del | 04C28139A* | 1.057 | 0.049 | 4562369091_21 | rs715106 | −0.093 |
| chr3: 4063809-4074877 | Del | 325927264 | 0.480 | 0.022 | 4240108555_11 | rs317528 | −0.508 |
| chr3: 4063809-4074877 | Del | 2577168153 | 0.452 | 0.006 | 1890578271_A | rs317528 | −0.607 |
| chr3: 4063809-4074877 | Del | 04C28087A* | 1.000 | 0.068 | 4562262038_21 | rs317528 | −0.040 |
| chr3: 4063809-4074877 | Del | 04C28139A* | 1.040 | 0.041 | 4562369091_21 | rs317528 | −0.028 |
| chr4: 9881886-9884092 | Del | 332702531 | 0.510 | 0.020 | 4290041726_12 | rs10939814 | −0.640 |
| chr4: 9881886-9884092 | Del | 6483240361 | 0.440 | 0.170 | 4243114252_11 | rs10939814 | −0.752 |
| chr4: 9881886-9884092 | Del | 9655625304 | 0.611 | 0.013 | 1837427556_A | rs10939814 | −0.585 |
| chr4: 9881886-9884092 | Del | 9966812554 | 0.482 | 0.024 | 4276098355_21 | rs10939814 | −0.502 |
| chr4: 9881886-9884092 | Del | 04C28087A* | 1.000 | 0.110 | 4562262038_21 | rs10939814 | −0.040 |
| chr4: 9881886-9884092 | Del | 04C28139A* | 0.823 | 0.025 | 4562369091_21 | rs10939814 | −0.059 |
| chr18: 38310567-38311765 | Del | 1317180605 | 0.000 | 0.000 | 4256206108_21 | rs10468964 | −4.483 |
| chr18: 38310567-38311765 | Del | 3613918399 | 0.000 | 0.000 | 4276098785_12 | rs10468964 | −4.855 |
| chr18: 38310567-38311765 | Del | 3673606183 | 0.000 | 0.000 | 4240108637_11 | rs10468964 | −4.646 |
| chr18: 38310567-38311765 | Del | 5301838910 | 0.000 | 0.000 | 4523280020_21 | rs10468964 | −4.984 |
| chr18: 38310567-38311765 | Del | 8334564658 | 0.000 | 0.000 | 4079300087_A | rs10468964 | −5.693 |
| chr18: 38310567-38311765 | Del | 04C28087A | 1.000 | 0.057 | 4562262038_21 | rs10468964 | −0.009 |
| chr18: 38310567-38311765 | Del | 04C28139A | 0.987 | 0.071 | 4562369091_21 | rs10468964 | 0.033 |

*Negative Control Samples (Normal Diploid)

To replicate the significant findings, we examined a replication cohort of 580 schizophrenia cases and 1,485 controls. Of the 25 significant loci in the discovery cohort, 8 were observed to be enriched in the cases of the replication cohort as well with nominal significance (Table 4). Among those, 5 loci were very rare in controls (<0.25%) while the other 3 presented common CNVs that were overrepresented in the cases. The resulting combined P-values ranged from 7.73×10⁻⁶ to 5.05×10⁻², for all CNVs in Table 4, of which four survive correction for 21 and 5 tests for deletion and duplication CNVRs respectively. Notably, two genes belong to the calcium signaling family (CACNA1B and DOC2A) and two other genes belong to the Ras signaling gene family (RET and RIT2), both of which are involved in neuronal development and signaling.

Although some genes did not replicate in our independent set of cases and controls of relatively modest size, these genes have supporting functional roles to schizophrenia and may replicate with further study of larger sample sizes. Additional Ras related cell cycle regulation family genes associated include: PTPRG, RAB23, TM2D3, SHC2, and RAPGEF2. PTBLP, RIN2, and RASA3 are also Ras genes supported by our genotype GWA presented in Table 6. Additional Calcium signaling family genes associated include: CAMK2D and KCNMB4. We also associate PARK2, RFWD2, and PTPRB, which we have previously associated with autism²¹, the latter interacting with the contactin gene family. These nominally significant loci may be singularly unconvincing, so we sought to identify the pathway perturbed in various ways by CNVs of different loci. Thus, we nominally associate an additional 5 Ras neural crest development genes and 2 calcium regulatory signaling genes for a total of 7 Ras genes and 4 Ras linked calcium-dependent signaling genes impacted with CNVs associated with schizophrenia. When taken together, the Gene Ontology (GO) class, synaptic transmission genes (CACNA1B, PARK2, KCNMB4, GJD2, DOC2A, COMT, RIT2, and ATXN1), was significantly enriched in the cases (P=1.5×10⁻⁷).

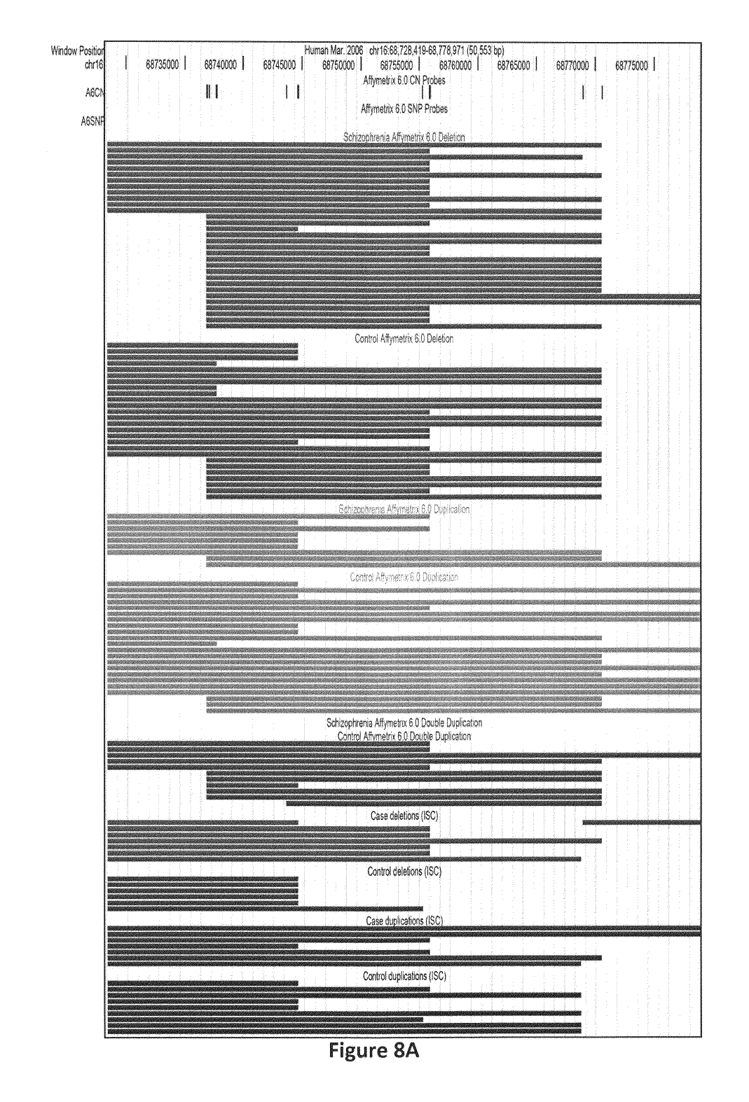

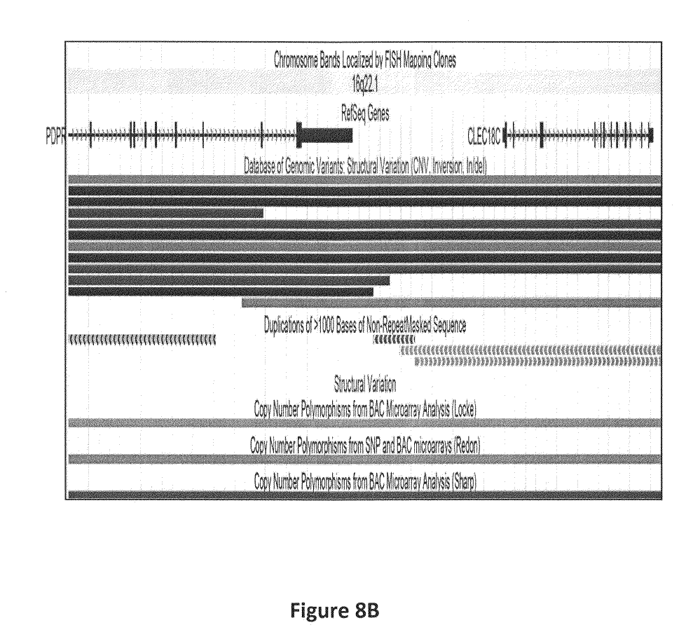

The genes impacted by or proximal to significant CNVs encode proteins with intriguing function. PDPR or pyruvate dehydrogenase phosphatase regulatory, is involved in glycine catabolism and the ISC data shows six novel deletions in cases and one in controls. In FIG. 8, we show that this locus replicated in 30 independent cases and direct impact of PDPR, using the UCSC Genome Browser²² with Build 36. The 22q11.21 deletion locus was previously reported in 11 cases and no controls by the International Schizophrenia Consortium (ISC)⁸, an association to schizophrenia previously reported and well supported⁴. Within 22q11.21, COMT catalyzes the transfer of a methyl group from S-adenosylmethionine to catecholamines, including the neurotransmitters dopamine, epinephrine, and norepinephrine. DOC2A is mainly expressed in brain and is involved in Ca(2+)-dependent neurotransmitter release. Observation of this large constitutional duplication (and deletion) was also observed in Autism cases^{41,42,21} CACNA1B is a N-type calcium channel, which controls neurotransmitter release from neurons. CACNA1C has been robustly associated with bipolar disorder based on genotypes of 4,387 cases and 6,209 controls ²³. One deletion and four duplications were found in cases while there was one control duplication over the span of CACNA1C. RET is a receptor tyrosine kinase, a cell-surface molecule that transduces signals for cell growth and differentiation, which plays a crucial role in neural crest development²⁴. RET loss of function is associated with Hirschsprung's disease, while gain of function is associated with cancer development. SUMF1 (UNQ3037) deletion was reported by us in 11 unrelated cases in association with autism²¹. SUMF1 catalyzes the hydrolysis of sulfate esters such as glycosaminoglycans, sulfolipids, and steroid sulfates. WDR1 is involved with actin formation and sensory perception of sound. Studies using shotgun mass spectrometry found it to be differentially expressed in the dorsolateral prefrontal cortex of schizophrenia patients²⁵. RIT2 is a Ras-like protein expressed in neurons. PIK3C3 has been shown to harbor a promoter mutation that increases the risk of schizophrenia and bipolar disorder.

Ras has been the focus of many cancer studies as a pivotal tumor suppressor but less emphasis has been placed on the native biological role of Ras for neuronal survival, differentiation, and plasticity. Ras is necessary for neurotrophin-induced neuronal survival. It is clear from in vitro models that calcium is required for activity-dependent potentiation of the strength of many synapses. Calcium-mediated pathways of Ras activation may be a critical mechanism to couple rapid and transient neuronal electrical activity with long-term changes in nervous system development and function²⁶⁻³⁰. Here we show that deletions of these genes, critical to brain development and function in ras and calcium pathways, predispose subjects to schizophrenia. Synaptic connectivity linking neurons and subsequent alteration may enable memory formation and behavior adaptation. Calcium influx into dendritic spines, termination point of excitatory synapses, is an activation switch for a myriad of signaling pathways important for synaptic plasticity. The small GTPase protein Ras couples calcium influx to many forms of synaptic plasticity, such as rapid synaptic potentiation and new synapse formation. Ras activation can also trigger protein synthesis and gene transcription important for the long-term maintenance of synaptic plasticity and for many other neuronal responses, including cell survival, death, and differentiation. Consistent with many essential roles of Ras signaling in neuronal plasticity, mutations in the Ras signaling pathway are associated with other diseases causing cognitive impairments and learning deficits such as autism, X-linked mental retardation and neurofibromatosis 1³¹⁻³³. Indeed, we have identified rare highly penetrant CNVs in ubiquitin genes and common CNVs that were overrepresented in neuronal development in autism²¹. Further, based on genotype association, a common variant on 5p14.1 between CDH10 and CDH9 encoding neuronal cell-adhesion molecules also associated with autism³⁴.

In conclusion, using a genome-wide approach for high-resolution CNV detection, we have identified candidate genomic loci with enrichment of CNVs in schizophrenia cases as compared to controls, and replicated many of them using an independent data set of schizophrenia cases and controls. Two genes impacted encode calcium signaling molecules (CACNA1B and DOC2A) and two other genes belong to the Ras signaling gene family (RET and RITZ), both of which are involved in neuronal development and signaling. Together, these genes show significant enrichment in the gene family of synaptic transmission molecules based on Gene Ontology (P=1.5×10⁻⁷). The enrichment of genes within this molecular system suggests novel susceptibility mechanisms for schizophrenia, and will spur identification of additional variations, including structural variations and single-base changes in candidates within these gene networks. In addition, our results call for functional expression assays to assess the biological effects of CNVs in these candidate genes in brain tissue.

### REFERENCE FOR EXAMPLE II

1. Arajarvi R. Prevalence and diagnosis of schizophrenia based on register, case record and interview data in an isolated Finnish birth cohort born 1940-1969. Soc Psychiatry Psychiatr Epidemiol. 40(10):808-16 (2005).
4. Liu, H. et al., Genetic variation in the 22q11 locus and susceptibility to schizophrenia Proc. Natl. Acad. Sci. U.S.A. 99, 16859-16864 (2002).
5. Kirov, G. et al., Comparative genome hybridization suggests a role for NRXN1 and APBA2 in schizophrenia. Hum. Mol. Genet. 17(3) 458-465 (2007).
6. Friedman, J. I. et al., CNTNAP2 gene dosage variation is associated with schizophrenia and epilepsy. Mol. Psychiatry Molecular Psychiatry 13 261-266 (2008).
7. Walsh, T. et al. Rare Structural Variants Disrupt Multiple Genes in Neurodevelopmental Pathways in Schizophrenia Science 320 539-543 (2008).
8. The International Schizophrenia Consortium. Rare chromosomal deletions and duplications increase risk of schizophrenia. Nature 455, 237-241 (2008).
9. Stefansson, H. et al. Large recurrent microdeletions associated with Schizophrenia Nature 455, 232-236 (2008).
10. Shi, Y. Y. et al. A study of rare structural variants in schizophrenia patients and normal controls from Chinese Han population. Molecular Psychiatry 13, 911-913 (2008).
11. Need, A. C., Dongliang, G., Weale, M. E., Maia, J., Feng, S., et al. (2009) A Genome-Wide

Investigation of SNPs and CNVs in Schizophrenia. PLoS Genet 5(2): e1000373 (2009). doi:10.1371/journal.pgen.1000373.
12. GAIN Collaborative Research Group et al. New models of collaboration in genome-wide association studies: the Genetic Association Information Network. Nat Genet. 39(9):1045-51 (2007).
13. Suarez, B. K. et al. Genomewide linkage scan of 409 European-ancestry and African American families with schizophrenia: suggestive evidence of linkage at 8p23.3-p21.2 and 11p13.1-q14.1 in the combined sample. Am J Hum Genet. 78(2), 315-33 (2006).
14. O'Donovan, M. C. et al. Identification of loci associated with schizophrenia by genome-wide association and follow-up. Nat Genet. 40(9),1053-5 (2008).
15. O'Donovan, M. C. et al. Analysis of 10 independent samples provides evidence for association between schizophrenia and a SNP flanking fibroblast growth factor receptor 2. Mol Psychiatry. 14(1):30-6 (2009).
16. Sanders, A. R. et al. No significant association of 14 candidate genes with schizophrenia in a large European ancestry sample: implications for psychiatric genetics. Am J Psychiatry. 165(4),497-506 (2008).
17. Shi, J. et al. Common variants on chromosome 6p22.1 are associated with schizophrenia. Nature advance online publication 1 Jul. 2009 I doi:10.1038/nature08192.
18. Flaum M. & Andreasen, N. C. Diagnostic Criteria for Schizophrenia and Related Disorders: Options for DSM-IV. Schizophrenia Bulletin 17, 133-142 (1991).
19. Redon, R. et al. Global variation in copy number in the human genome Nature 444, 444-454 (2006).
20. Wang, K. et al. PennCNV: an integrated hidden Markov model designed for high-resolution copy number variation detection in whole-genome SNP genotyping data. Genome Res. 17, 1665-1674 (2007).
21. Glessner, J. T. et al., Autism genome-wide copy number variation reveals ubiquitin and neuronal genes. Nature 459, 569-573 (2009).
22. Kent W. J et al. The human genome browser at UCSC. Genome Res. 12(6), 996-1006 (2002).
23. Ferreira, Manuel A R Collaborative genome-wide association analysis supports a role for ANK3 and CACNA1C in bipolar disorder. Nature Genetics 40, 1056-1058 (2008).
24. Lia, L. et al. The role of Ret receptor tyrosine kinase in dopaminergic neuron development. Neuroscience 142(2), 391-400 (2006).
25. Martins-de-Souza D., et al. Prefrontal cortex shotgun proteome analysis reveals altered calcium homeostasis and immune system imbalance in schizophrenia. Eur Arch Psychiatry Clin Neurosci. 259(3) (2009).
26.Farnsworth, C. L. et al. Calcium activation of Ras mediated by neuronal exchange factor Ras-GRF. Nature. 376(6540), 524-7 (1995).
27. Finkbeiner, S. & Greenberg, M. E. Ca2⁺-Dependent Routes to Ras: Mechanisms for Neuronal Survival, Differentiation, and Plasticity? Neuron 16, 233-236 (1996)
28. Oh, J. S., Manzerra, P., & Kennedy, M. B. Regulation of the Neuron-specific Ras GTPase-activating Protein, synGAP, by Ca2+/Calmodulin-dependent Protein Kinase II. J. Biol. Chem. 279(17), 17980-17988 (2004).
29. Yoshimuraa, T., et al. Ras regulates neuronal polarity via the PI3-kinase/Alct/GSK-3β/CRMP-2 pathway. Biochemical and Biophysical Research Communications 340(1) 62-68 (2006).
30. Yoshimura T., Arimura N., & Kaibuchi K. Signaling Networks in Neuronal Polarization. The Journal of Neuroscience, 26(42), 10626-10630 (2006).
31. Antonarakis, S. E. & Van Aelst, L., Nat. Genet. 19, 106-108 (1998).
32. Chelly, J. & Mandel, J. L., Mind the GAP, Rho, Rab and GDI. Nat. Rev. Genet. 2, 669-680 (2001).
33. Comings, D. E, Wu, S., Chiu, C., Muhleman, D. & Sverd, J. Studies of the c-Harvey-Ras gene in psychiatric disorders. Psychiatry Res. 63, 25-32 (1996).
34. Wang K. et al. Common genetic variants on 5p14.1 associate with autism spectrum disorders. Nature 459, 528-533 (2009).
35. Diskin, S. et al. Adjustment of genomic waves in signal intensities from whole-genome SNP genotyping platforms. Nucleic Acids Research. 36(19) (2008).
36. G Dennis Jr et al. DAVID: Database for Annotation, Visualization, and Integrated Discovery. Genome Biology. 4(9), (2003).
37. Lencz T. Runs of homozygosity reveal highly penetrant recessive loci in schizophrenia PNAS. 104 19942-19947 (2007).
38. Xu, B. et al. Strong association of de novo copy number mutations with sporadic schizophrenia Nature Genetics 40, 880-885 (2008).
39. Edmondson A. et al Loss-of-function variants in endothelial lipase are a cause of elevated HDL cholesterol in humans. J Clin Invest. 119(4): 1042-1050. (2009).
40. Lehrke M, et al CXCL16 is a marker of inflammation, atherosclerosis, and acute coronary syndromes in humans. J Am Coll Cardiol. 49(4), 442-9 (2007).
41. Weiss, L. A. et al. Association between microdeletion and microduplication at 16p11.2 and autism. N. Engl. J. Med. 358, 667-675 (2008).
42. Kumar, R. A. et al. Recurrent 16p11.2 microdeletions in autism. Hum. Mol. Genet. 17, 628-638 (2008).

### EXAMPLE III

### Screening Assays for Identifying Efficacious Therapeutics for the Treatment of Schizophrenia

The information herein above can be applied clinically to patients for diagnosing an increased susceptibility for developing schizophrenia and for therapeutic intervention. A preferred embodiment of the invention comprises clinical application of the information described herein to a patient. Diagnostic compositions, including microarrays, and methods can be designed to identify the genetic alterations described herein in nucleic acids from a patient to assess susceptibility for developing schizophrenia. This can occur after a patient arrives in the clinic; the patient has blood drawn, and using the diagnostic methods described herein, a clinician can detect a CNV shown in Tables 2, 3, 4, 5 and 7. The information obtained from the patient sample, which can optionally be amplified prior to assessment, will be used to diagnose a patient with an increased or decreased susceptibility for developing schizophrenia. Kits for performing the diagnostic method of the invention are also provided herein. Such kits comprise a microarray comprising at least one of the CNVs provided herein in and the necessary reagents for assessing the patient samples as described above.

In accordance with the present invention, it has been found that a certain percentage of patients with schizophrenia carry specific types of mutations of genes that encode for metabotropic glutamate receptors (mGluRs). These mutations are sensitive and specific biomarkers for selecting and treating schizophrenia due to defective mGluR pathways. Furthermore, the present inventors have identified drug candidates that specifically activate the mGluRs, potentially restoring normal neurophysiology in schizophrenia patients harboring mutations in the GRM family of mGluR genes.

Compounds which may be administered in implementing the test and treat paradigm described herein include the piracetam family of nootropic agents, as described in F. Gualtieri et al., Curr. Pharm. Des., 8: 125-38 (2002). More preferably, the treating agent is a pyroglutamide. Details regarding the preparation and formulation of pyroglutamides which may be used in the practice of this invention are provided in U.S. Pat. No. 5,102,882 to Kimura et al. A particularly preferred agent for the treatment of schizophrenia in patients determined to have one or more of the SNPs indicative of the presence of an schizophrenia associated copy number variation, as set forth in the tables herein, is (+)-5-oxo-D-prolinepiperidinamide monohydrate (NS-105). A variety of pyroglutamide derivatives (see, e.g., U.S. Pat. No. 5,102,882) and other members of the piracetam family of nootropic agents are currently available. Such agents should also have utility for the treatment of schizophrenia as described hereinabove.

The identity of schizophrenia- involved genes and the patient results will indicate which variants are present, and will identify those that possess an altered risk for developing schizophrenia. The information provided herein allows for therapeutic intervention at earlier times in disease progression than previously possible. Also as described herein above, the genes containing the CNVs of the invention provide novel target for the development of new therapeutic agents efficacious for the treatment of this neurological disease.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for detecting a propensity for developing schizophrenia, the method comprising: detecting the presence of at least one deletion containing CNV in a target polynucleotide wherein if said CNV is present, said patient has an increased risk for developing schizophrenia, wherein said deletion containing CNV is selected from the group of CNVs consisting of chr1:194097653-194148082, chr12:84799874-84809923, chr19:471192213-47196345, chr3:60564450-60565103, chr5:78285889-78300897, chr13:81402686-81416252 and chr11:88016449-88023261.

2. A method for detecting a propensity for developing schizophrenia, the method comprising: detecting the presence of at least one duplication containing CNV in a target polynucleotide wherein if said CNV is present, said patient has an increased risk for developing schizophrenia, wherein said duplication containing CNV is selected from the group of CNVs consisting of chr8:26404795-26404795, chr1:174500555-174543675 and chr12:18801189-18821605.

3. The method as claimed in claim 1 or 2, wherein the target nucleic acid is amplified prior to detection.

4. The method of claim 1 or 2, wherein the step of detecting the presence of said CNV is performed using a process selected from the group consisting of detection of specific hybridization, measurement of allele size, restriction fragment length polymorphism analysis, allele-specific hybridization analysis, single base primer extension reaction, and sequencing of an amplified polynucleotide.

5. The method as claimed in claim 1 or 2, wherein in the target nucleic acid is DNA.

6. The method of claim 1 or 2, wherein nucleic acids comprising said CNV are obtained from an isolated cell of the human subject.

7. A method for identifying therapeutic agents which alter neuronal signaling and/or morphology, comprising
a) providing cells expressing at least one CNV as claimed in claim 1 or claim 2;
b) providing cells which express the cognate wild type sequences corresponding to the CNV of step a);
c) contacting the cells of steps a) and b) with a test agent and
d) analyzing whether said agent alters neuronal signaling and/or morphology of cells of step a) relative to those of step b), thereby identifying agents which alter neuronal signaling and morphology.

8. The method of claim 7 wherein said agent has efficacy for the treatment of schizophrenia or other related neurodevelopmental disorders.

9. A method for the treatment of schizophrenia in a patient in need thereof comprising administration of an effective amount of the agent identified by claim 7, said agent being contained within a pharmaceutically acceptable carrier.

10. The method of claim 8, wherein said agent modulates neuronal cell signaling.

11. A vector comprising at least one of the CNV-containing nucleic acids of claim 1 or claim 2.

12. A host cell comprising the vector of claim 11.

13. A solid support comprising the schizophrenia related CNV containing nucleic acid of claim 1 or claim 2.

14. A method for detecting a propensity for developing schizophrenia, the method comprising: detecting the presence of at least one CNV in a target polynucleotide wherein if said CNV is present, said patient has an increased risk for developing schizophrenia, wherein said CNV is selected from the group of CNVs consisting of
chr16:68743639-68770545,
chr22 :17404806-19941349,
chr16:29425212-30134444,
chr9:140145139-140152969,
chr10:42932615-42934354,
chr3 :4063809-4074877,
chr4:9881886-9884092, and
chr18:38310567-38311765.

15. The method of claim 14, wherein said CNV contain a deletion in a gene selected from the group consisting of PDPR, COMT, CACNA1B, RET, SUMF1, WDR1, RIT2 and PIK3C3.

16. The method of claim 14, wherein said CNV contains a duplication in a gene selected from the group consisting of QPRT, DOC2A and TBX6.

17. An isolated nucleic acid comprising at least one CNV as claimed in claim 14, optionally contained in an expression vector.

18. A method for detecting a propensity for developing schizophrenia, the method comprising: detecting the presence of at least one CNV in a target polynucleotide wherein if said CNV is present, said patient has an increased risk for developing schizophrenia, wherein said CNV is selected from the group of CNVs consisting of
chr7:32177451-32392975,
chr3:61803641-61811383,
chr4:135276704-135408238,
chr5:2097129-2111366,
chr12:60558836-60563972,
chr6:57268143-57272458,
chr5:52702915-52718131,
chr19:426716-434473,
chr6:16499554-16508717,
chr15:99980078-100033288,
chr15:32717247-32765105,
chr7:142941348-142963649,
chr4:114573691-114581335,
chr4:162417655-162424561,
chr6:162740476-162741040,
chr1:92014319-92021028, and
chr12:69158942-69164294.

19. An isolated nucleic acid comprising at least one CNV as claimed in claim 18, optionally contained in an expression vector.

20. A method for detecting a propensity for developing schizophrenia, the method comprising: detecting the presence of at least one CNV in a target polynucleotide wherein if said CNV is present, said patient has an increased risk for developing schizophrenia, wherein said CNV is selected from the group of CNVs consisting of
chr22:17404806-19941349 Del,
chr22:17404806-19941349 Del,
chr22:17404806-19941349 Del,
chr22:17404806-19941349 Del,
chr16:29425212-30134444 Dup,
chr16:29425212-30134444 Dup,
chr16:29425212-30134444 Dup,
chr16:29425212-30134444 Dup,
chr16:29425212-30134444 Dup,
chr16:68743639-68770545 Del,
chr16:68743639-68770545 Del,
chr16:68743639-68770545 Del,
chr9:140145139-140152969 Del,
chr9:140145139-140152969 Del,
chr9:140145139-140152969 Del,
chr9:140145139-140152969 Del,
chr9:140145139-140152969 Del,
chr9:140145139-140152969 Del,
chr9:140145139-140152969 Del,
chr9:140145139-140152969 Del,
chr9:140145139-140152969 Del,
chr10:42932615-42934354 Del,
chr10:42932615-42934354 Del,
chr10:42932615-42934354 Del,
chr10:42932615-42934354 Del,
chr10:42932615-42934354 Del,
chr10:42932615-42934354 Del,
chr10:42932615-42934354 Del,
chr3:4063809-4074877 Del,
chr3:4063809-4074877 Del,
chr3:4063809-4074877 Del,
chr3:4063809-4074877 Del,
chr4:9881886-9884092 Del,
chr4:9881886-9884092 Del,
chr4:9881886-9884092 Del,
chr4:9881886-9884092 Del,
chr4:9881886-9884092 Del,
chr4:9881886-9884092 Del,
chr18:38310567-38311765 Del,
chr18:38310567-38311765 Del,
chr18:38310567-38311765 Del,
chr18:38310567-38311765 Del,
chr18:38310567-38311765 Del,
chr18:38310567-38311765 Del, and
chr18:38310567-38311765 Del.

21. An isolated nucleic acid comprising at least one CNV as claimed in claim 20, optionally contained in an expression vector.

22. A method of treating schizophrenia in a human subject determined to have at least one single nucleotide polymorphism (SNP) indicative of the presence of a schizophrenia associated copy number variation, said at least one SNP being selected from the group consisting of CNVs of claim 20, the method comprising administering to said human subject a therapeutically effective amount of at least one member of the piracetam family of nootropic agents.

23. The method of claim 22, wherein said SNP is a deletion in at least one of the following: glutamate receptor, metabotropic 5 (GRM 5), glutamate receptor, metabotropic 7 (GRM 7), glutamate receptor, metabotropic 8 (GRM 8).

24. The method of claim 22, wherein said SNP is a duplication of glutamate receptor, metabotropic 1.

25. The method of claim 22, wherein said nootropic agent is a pyroglutamide.

26. The method of claim 25, wherein said pyroglutamide is (+)-5-oxo-D-prolinepiperidinamide monohydrate (NS-105).
